(19) United States
(12) Patent Application Publication
(10) Pub. No.: US 2009/0258869 A1
(43) Pub. Date: Oct. 15, 2009

(54) METHODS AND COMPOUNDS FOR TREATMENT OR PREVENTION OF SUBSTANCE-RELATED DISORDERS

(75) Inventors: Dorit RON, San Francisco, CA (US); Dao-Yao He, Hercules, CA (US)

Correspondence Address: JONES DAY, 222 EAST 41ST ST, NEW YORK, NY, 10017, US

(73) Assignee: The Regents of the University of California

(21) Appl. No.: 12/367,412

(22) Filed: Feb. 6, 2009

Related U.S. Application Data
(60) Provisional application No. 61/027,399, filed on Feb. 8, 2008.
(60) Provisional application No. 61/029,229, filed on Feb. 15, 2008.

(51) Int. Cl. A61K 31/5365; A61K 31/4985; A61K 31/4188

(52) U.S. Cl. 514/229.5; 514/250; 514/395

(57) ABSTRACT

The present disclosure provides methods of treating or preventing a substance-related disorder using Hsp90 inhibitors, Hsp90 modulators, tyrosine hydroxylase modulators, and modulators that reduce the interaction between Hsp90 and tyrosine hydroxylase.

### 1. CROSS-REFERENCE TO RELATED APPLICATIONS

This application claims the benefit under 35 U.S.C. § 119(e) of U.S. Provisional Application No. 61/027,399, filed Feb. 8, 2008, and U.S. Provisional Application No. 61/029,229, filed Feb. 15, 2008, all of which are incorporated herein by reference in their entirety.

### 2. GOVERNMENT SUPPORT

The research leading to this invention was supported, at least in part, with funding provided by Grant No. W81XWH-07-1-0079 from the United States Army and the Department of Defense, Grant No. RO1 AA014366 from the National Institute of Health and the National Institute on Alcohol Abuse and Alcoholism, and a grant from the State of California. The government may have certain rights to the invention.

### 3. FIELD OF THE INVENTION

The present disclosure provides methods of treating or preventing a substance-related disorder using Hsp90 inhibitors, Hsp90 modulators, tyrosine hydroxylase modulators, and modulators that reduce the interaction between Hsp90 and tyrosine hydroxylase.

### 4. BACKGROUND OF THE INVENTION

Drug- and substance-related disorders include disorders caused by various types of addictive (dependence-producing) substances. The number of patients/drug abusers suffering from substance-related disorders is estimated to exceed 30 million worldwide. The cost to society is high when social problems caused by substance-related mental disorder and socially dysfunctional characteristics of substance dependent individuals are considered. Thus there is a worldwide demand for an effective cure for substance-related disorders.

At the onset of substance-related disorder, the involvement of the CNS reward system has been elucidated. The reward system has been identified as the site responsible for intracranial self stimulation-related behaviors in animals and plays a role in eliciting senses of pleasure, motivation, and euphoria. The treatment of drug dependence can be made very difficult since many addictive substances have an activity of stimulating this system, thereby eliciting senses of pleasure in users, and the influence of such activity remains even after the drug, as a causative agent, is depleted from the body.

Most addictive drugs can cause dependence after a single administration. Once the user is affected, the symptoms sometimes persist over a long term even after the use is terminated. For these reasons, drug dependence is considered a chronic neurological disorder. Furthermore, overdose of such drugs may have a deteriorating effect on living body and may even cause death.

At present, there are few effective cures for substance-related disorders. For example alcohol dependence constitutes one of the most serious public health problems worldwide. There are only three medications available for the treatment of alcohol dependence: disulfiram, acamprosate and naltrexone. The opioid antagonist, naltrexone has demonstrated the most consistent effect in reducing alcohol consumption in the context of behavioral therapy (Anton et al., Jama 2006, 295, 2003-17). Naltrexone has been shown to decrease ethanol consumption in numerous animal studies (Altshuler et al., Life Sci. 1980, 26, 679-88; Froehlich et al., Pharmacol. Biochem. Behav. 1990, 35, 385-90; Stromberg et al., Alcohol Clin. Exp. Res. 1998, 22, 2186-91; Stromberg et al., Alcohol 2001, 23, 109-16; Volpicelli et al., Life Sci. 1986, 38, 841-7) and clinical studies (Anton et al., J. Clin. Psychopharmacol. 2001, 21, 72-7; O'Malley et al., Arch. Gen. Psychiatry 1992, 49, 881-7; Oslin et al., Am. J. Geriatr. Psychiatry 1997, 5, 324-32; Volpicelli et al., Arch. Gen. Psychiatry 1992, 49, 876-80) and has been shown to be more effective in heavy or excessive drinkers (Pettinati et al., J. Clin. Psychopharmacol. 2006, 26, 610-25). However, not all patients respond to naltrexone; this can be partly explained by genetic variations in the mu opioid receptor gene (Oslin et al., Addict. Biol. 2006, 11, 397-403). Furthermore, opioid receptor antagonists decrease both ethanol and sucrose intake in rodents (Beczkowska et al., Brain Res. 1992, 589, 291-301; Stromberg et al., Pharmacol. Biochem. Behav. 2002, 72, 483-90,). Alcohol dependence is a complex disorder that will require the use of different therapeutic approaches to effectively treat the disease.

Clearly, there remains a need for improved therapies for alcohol abuse and dependency as well as for substance-related disorders in general.

### 5. SUMMARY OF THE INVENTION

The present invention provides methods of treating or preventing of a substance-related disorder in a subject in need thereof comprising administering to the subject an amount of an Hsp90 inhibitor or modulator or tyrosine hydroxylase (“TH”) modulator, or modulators that reduce the interaction between Hsp90 and tyrosine hydroxylase or a pharmaceutically acceptable salt thereof, effective to treat or prevent the substance-related disorder.

In one aspect provided herein are methods of ameliorating or eliminating an effect of a substance-related disorder in a subject in need thereof, comprising administering to the subject an amount of an Hsp90 inhibitor or modulator or tyrosine hydroxylase modulator or modulators that reduce the interaction between Hsp90 and tyrosine hydroxylase or a pharmaceutically acceptable salt thereof, effective to ameliorate or eliminate the effect of the substance-related disorder.

In one aspect provided herein are methods for diminishing, inhibiting or eliminating an addiction-related behavior in a subject suffering from a substance-related disorder, comprising administering to the subject an amount of an Hsp90 inhibitor or modulator or tyrosine hydroxylase modulator or modulators that reduce the interaction between Hsp90 and tyrosine hydroxylase or a pharmaceutically acceptable salt thereof, effective to diminish, inhibit or eliminate the addiction-related behavior.

In one aspect provided herein are methods for alleviating or eliminating withdrawal symptoms in a subject suffering from a substance-related disorder comprising administering to the subject an amount of an Hsp90 inhibitor or modulator or tyrosine hydroxylase modulator or modulators that reduce the interaction between Hsp90 and tyrosine hydroxylase or a pharmaceutically acceptable salt thereof, effective to alleviate or eliminate the withdrawal symptoms.

### 6. BRIEF DESCRIPTION OF DRAWINGS

FIG. 1: Ethanol induces an increase in TH immunoreactivity in a time- and dose-independent manner. (A) SH-SY5Y cells were treated without (lane 1, control) or with 100 mM ethanol (lanes 2-4) for the indicated times. TH protein levels were analyzed by Western blot with anti-TH antibody and actin protein levels were detected with anti-Actin antibody as an internal control. Histogram depicts the mean percentage change in the ratio of TH to Actin+/−SD from seven experiments. (B) Cells were treated for 24 h without (lane 1, control) or with different concentrations of ethanol (lanes 2-4). Histogram depicts the mean percentage change in the ratio of TH to Actin+/−SD from three experiments. (C) Cells were treated without (lane 1, control) or with 100 mM ethanol for 24 h (lane 2), or with 100 mM ethanol for 24 h followed by washing with media and incubation without ethanol for an additional 24 h (lane 3). Histogram depicts the mean percentage change in the ratio of TH to Actin+/−SD from four experiments. *, p<0.05; **, p<0.01, compared to lane 1 (control).

FIG. 2: Chronic ethanol does not increase the transcription and translation of TH, but enhances TH protein stability. (A) Cells were treated without (lane 1, control) or with 100 mM ethanol for 12 h or 24 h (lanes 2 & 3). TH mRNA expression levels were analyzed by RT-PCR with Actin mRNA levels as an internal control. Histogram depicts the mean percentage change in the ratio of TH to Actin+/−SD from three experiments. (B) Cells were treated without (lane 1, control, & lane 3) or with 100 mM ethanol (lanes 2 & 4) for 24 h, and 30 μg/ml cycloheximide (CHX) was added as indicated for the last 12 h of the 24-h treatment (lanes 3 & 4). TH protein levels were analyzed as described above. Histogram depicts the mean percentage change in the ratio of TH to Actin+/−SD from three experiments. **, P<0.01, compared to control. (C) Cells were treated without (control) or with 100 mM ethanol for 24 h before a pulse-chase procedure as described in Experimental Procedures. Cells were labeled with 25 μCi ³⁵S-Met/Cys pro-mix cell labeling mix for 3 h and then incubated in the normal medium for the indicated chase times, followed by immunoprecipitation with anti-TH antibody, separation on an SDS-PAGE gel and autoradiography. Histogram depicts the mean percentage change in radioactive signals of ³⁵S-labelled TH protein at each chase time to those at initiation of chase (0 time) from six experiments. *, p<0.05; **, p<0.01, compared to control.

FIG. 3. Geldanamycin, an inhibitor of heat shock protein 90, inhibits chronic ethanol-induced TH accumulation. (A) Cells were treated without (lane 1) or with 100 mM ethanol for 24 h (lane 2), or with 100 mM ethanol for 24 h to which different doses of geldanamycin (GA) were added for the last 9 h of ethanol treatment (lanes 3-5). TH protein levels were analyzed as described above. Histogram depicts the mean percentage change in the ratio of TH to Actin+/−SD from three experiments. *, p<0.05, lanes 2 vs. 1, or 4 vs. 2; **, p<0.01, lanes 5 vs. 2. (B) Cells were treated without (Con), or with 100 mM ethanol for 12 h (Et 12 h) or 24 h (Et 24 h), or 100 mM ethanol for 24 h in which 1 μM GA was added for the last 9 h (Et 24 h/GA 9 h). Protein levels of Hsp90 and Actin were measured by Western blot analysis. Histogram depicts the mean ratio of Hsp90 to Actin+/−SD from three experiments.

FIG. 4. Chronic ethanol induces an association of TH with heat shock protein 90. (A & B) Cells were treated without (Con) or with 100 mM ethanol for 24 h (Et), or with 100 mM ethanol for 24 h to which 1 μM GA was added for the last 9 h (Et/GA). TH was co-immunoprecipitated with Hsp90 using anti-Hsp90 antibody (A) and Hsp90 was co-immunoprecipitated with TH using anti-TH antibody (B). Hsp90 and TH levels in input samples were analyzed by Western blot. Images are representative of four experiments. (C) Cells were treated without (Con) or with 100 mM ethanol for 24 h (Et). Akt was co-immunoprecipitated with Hsp90 using anti-Hsp90 antibody (left panel) and Hsp90 was co-immunoprecipitated with Akt using anti-Akt antibody (right panel). Images are representative of three experiments.

FIG. 5. GDNF decreases ethanol-mediated stabilization and association of TH with Hsp90. (A) Cells were treated with 100 mM ethanol for 24 h alone (Et/-GDNF) or together with 25 ng/ml GDNF added for the last 12 h (Et/+GDNF) before a pulse-chase procedure as described in Example 1 with the chase times as indicated. Histogram depicts the mean percentage change in radioactive signals of ³⁵S-labelled TH protein from three experiments. *, p<0.05. (B) Cells were treated without (Con) or with 100 mM ethanol for 24 h alone (Et) or in combination with 25 ng/ml GDNF added for the last 12 h (Et/GDNF). Co-immunoprecipitation of Hsp90 with TH was analyzed as described in Example 1. Image is representative of three experiments.

FIG. 6. Cells were treated without (lane 1) or with 10 μM Ibogaine for 12 h (lane 2), 100 mM ethanol for 24 h (lane 3) or 100 mM ethanol for 24 h to which 10 μM Ibogaine was added for the last 12 h of ethanol incubation (lanes 4-6): Lane 4, ethanol plus ibogaine, Lane 5, ethanol plus ibogaine after 1-h preincubation with PI-PLC (see Experimental Procedures), Lane 6, ethanol plus ibogaine together with 10 μg/ml of anti-GDNF neutralizing antibodies. Histogram depicts the mean percentage change in the ratio of TH to Actin+/−SD from three experiments. *, p<0.05; **, p<0.01.

FIG. 7. An Hsp90 inhibitor, 17-AAG, was systemically administered to rats, and voluntary alcohol intake and preference was measured. A 20% ethanol intermitten-access paradigm in which the rats consume large quantities of alcohol was used. Administration of 17-AAG (50 mg/kg), significantly reduced ethanol consumption (g/kg/24 hrs) and preference for ethanol (%), as compared to administration of vehicle. Water consumption was unaltered as shown by total fluid intake (ml/kg/24 hrs).

### 7. DETAILED DESCRIPTION OF THE INVENTION

### 7.1 Definitions

As used herein, the following terms shall have the following meanings:

The terms “treat,” “treating” or “treatment,” as used herein, refer to a method of alleviating or abrogating a disorder and/or its attendant symptoms. The terms “prevent,” “preventing” or “prevention,” in certain embodiments, refer to a method of barring a subject from acquiring a disorder and/or its attendant symptoms. In certain embodiments, the terms “prevent,” “preventing,” or “prevention,” refer to a method of reducing the risk of acquiring a disorder and/or its attendant symptoms.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington. The Science and Practice of Pharmacy, 21 st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

The term “subject” refers to animals such as mammals, including, but not limited to, primates (e.g., humans), cows, sheep, goats, horses, dogs, cats, rabbits, rats, mice and the like. In preferred embodiments, the subject is a human.

The term “substance-related disorder” refers to a Substance Use Disorder known to practitioners of skill in the art such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); a Caffeine Related Disorder such as Caffeine Intoxication (305.90), Caffeine-Induced Anxiety Disorder, Caffeine-Induced Sleep Disorder and Caffeine-Related Disorder Not Otherwise Specified (292.9); a Cannabis-Related Disorder such as Cannabis Dependence (304.30), Cannabis Abuse (305.20), Cannabis Intoxication (292.89), Cannabis Intoxication Delirium, Cannabis-induced Psychotic Disorder, Cannabis-induced Anxiety Disorder and Cannabis-Related Disorder Not Otherwise Specified (292.9); a Cocaine-Related Disorder such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); an Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-induced Psychotic Disorder, Opioid-induced Mood Disorder, Opioid-induced Sexual Dysfunction, Opioid-induced Sleep Disorder and Opioid-Related Disorder Not Otherwise Specified (292.9); a Phencyclidine (or Phencyclidine-Like)-Related Disorder such as Phencyclidine Dependence (304.60), Phencyclidine Abuse (305.90), Phencyclidine Intoxication (292.89), Phencyclidine Intoxication Delirium, Phencyclidine-induced Psychotic Disorder, Phencyclidine-induced Mood Disorder, Phencyclidine-induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-induced Psychotic Disorder, Sedative-, Hypnotic-, or Anxiolytic-induced Mood Disorder, Sedative-, Hypnotic-, or Anxiolytic-induced Anxiety Disorder Sedative-, Hypnotic-, or Anxiolytic-induced Sexual Dysfunction, Sedative-, Hypnotic-, or Anxiolytic-induced Sleep Disorder and Sedative-, Hypnotic-, or Anxiolytic-Related Disorder Not Otherwise Specified (292.9); Polysubstance-Related Disorder such as Polysubstance Dependence (304.80); and another (or Unknown) Substance-Related Disorder induced by Anabolic Steroids, Nitrate Inhalants and Nitrous Oxide. The terms describing the indications used herein are classified in the Diagnostic and Statistical Manual of Mental Disorders, 4th Edition, published by the American Psychiatric Association (DSM-IV); the “Diagnostic and Statistical Manual of Mental Disorders, Fourth Edition, Text Revision (DSM-IV-TR)”, Washington, D.C. 1 American Psychiatric Association, 2000; and/or the International Classification of Diseases, 10th Edition (ICD-10). The contents of all are hereby incorporated by reference in their entireties. The various subtypes of the disorders mentioned herein are contemplated as part of the present invention. Numbers in brackets after the listed diseases above refer to the classification code in DSM-IV.

The term “substance” as used herein refers to a substance that causes a substance-related disorder. Substances include, but are not limited to alcohol, amphetamine or similarly acting sympathomimetics, caffeine, cannabis, cocaine, hallucinogens, inhalants, nicotine, opioids, phencyclidine (PCP) or similarly acting arylcyclohexylamines, sedatives, hypnotics, anxiolytics or medications such as anesthetics, analgesics, anticholinergic agents, anticonvulsants, antihistamines, antihypertensive and cardiovascular medications, antimicrobial medications, anti-parkinsonian medications, chemotherapeutic agents, corticosteroids, gastrointestinal medications, muscle relaxants, nonsteroidal anti-inflammatory medications, other over-the-counter medications, antidepressant medications, and disulfiram. In another embodiment substances which can lead to the development of a substance-related disorder are toxic substances such as but not limited to heavy metals (e.g., lead or aluminium) rat poisons containing strychnine, pesticides containing nicotine, or acetylcholine-esterase inhibitors, nerve gases, ethylene glycol (antifreeze), carbon monoxide, and carbon dioxide. In yet another embodiment substances which can lead to the development of a substance-related disorder are volatile substances or “inhalants” (e.g., fuel, paint) if they are used for the purpose of becoming intoxicated; they are considered toxins if exposure is accidental or part of intentional poisoning.

The term “withdrawal” as used herein refers to the development of a substance-specific maladaptive behavioral change, with physiological and cognitive concomitants, that is due to the cessation of, reduction in, heavy and prolonged substance use. This substance-specific syndrome can cause clinically significant distress or impairment in social, occupational, or other important areas of functioning. The symptoms are not due to a general medical condition and are not accounted for by any other mental disorder. Withdrawal is usually, but not always, associated with Substance Dependence. Most (perhaps all) individuals with Withdrawal have a craving to re-dminister the substance to reduce the symptoms. The diagnosis of Withdrawal is recognized, but not limited to the following groups of substances: alcohol; amphetamines and other related substances; cocaine; nicotine; opioids; and sedatives, hypnotics, and anxiolytics. The dose and duration of use and other factors such as the presence or absence of additional illnesses also affect withdrawal symptoms.

The term “addiction-related behavior” as used herein refers to behavior resulting from compulsive substance use and is characterized by apparent substance dependency.

The term “substance dependency” or “substance dependence” as used herein refers to a condition of a subject displaying a maladaptive pattern of substance use, leading to clinically significant impairment or distress, as manifested by three (or more) of the following apparent to a practitioner of skill in the art, occurring any time in the same 12-month period:
(1) tolerance, as defined by either of the following:
   (a) a need for markedly increased amounts of the substance to achieve intoxication or desired effect
   (b) markedly diminished effect with continued use of the same amount of the substance
(2) withdrawal, as manifested by either of the following:
   (a) the characteristic withdrawal syndrome for the substance (development of a substance-specific syndrome due to the cessation of (or reduction in) substance use that has been heavy and prolonged, wherein the substance-specific syndrome causes clinically significant distress or impairment in social, occupational, or other important areas of functioning)
   (b) the same (or a closely related) substance is taken to relieve or avoid withdrawal symptoms
(3) the substance is often taken in larger amounts or over a longer period than was intended
(4) there is a persistent desire or unsuccessful efforts to cut down or control substance use
(5) a great deal of time is spent in activities necessary to obtain the substance (e.g. visiting multiple doctors or driving long distances), use the substance (e.g. chain smoking), or recover from its effects
(6) important social, occupational, or recreational activities are given up or reduced because of substance use
(7) the substance use is continued despite the knowledge of having a persistent or recurrent physical or psychological problem that is likely to have been caused or exacerbated by the substance (e.g. current cocaine use despite recognition of cocaine induced depression, or continued drinking despite recognition that an ulcer was made worse by alcohol consumption)

The term “alcohol” and “ethanol” as used herein are interchangeable.

The term “alcohol abuse” as used herein refers to a condition of a subject displaying a maladaptive pattern of alcohol use leading to clinically significant impairment or distress, as manifested by one (or more) of the following apparent to a practitioner of skill in the art occurring within a 12-month period: recurrent alcohol use resulting in a failure to fulfill major role obligations at work, school, or home (e.g., school and job performance may suffer either from the aftereffects of drinking or from actual intoxication on the job or at school; child care or household responsibilities may be neglected; and alcohol-related absences may occur from job or school); recurrent alcohol use in situations in which it is physically hazardous (e.g., driving an automobile or operating machinery while intoxicated); recurrent alcohol-related legal problems (e.g., arrests for intoxicated behavior or for driving under the influence); continued alcohol use despite having persistent or recurrent social or interpersonal problems caused or exacerbated by the effects of the substance (e.g., violent arguments with spouse while intoxicated, child abuse). Alcohol abuse requires fewer symptoms and, thus, may be less severe than dependence and is only diagnosed once the absence of dependence has been established.

The term “alcohol withdrawal” as used herein refers to a condition of a subject fulfilling the following diagnostic criteria as judged by a practitioner of skill in the art:
(1) Cessation of (or reduction in) alcohol use that has been heavy and prolonged.
(2) Two (or more) of the following, developing within several hours to a few days after Criterion (1):
   (a) autonomic hyperactivity (e.g., sweating or pulse rate greater than 100)
   (b) increased hand tremor
   (c) insomnia
   (d) nausea or vomiting
   (e) transient visual, tactile, or auditory hallucinations or illusions
   (f) psychomotor agitation
   (g) anxiety
   (h) grand mal seizures
(3) The symptoms in Criterion (2) cause clinically significant distress or impairment in social, occupational, or other important areas of functioning.
(4) The symptoms are not due to a general medical condition and are not better accounted for by another mental disorder.

The term “Hsp90 inhibitor” as used herein refers to a compound that disrupts the structure and/or function of an Hsp90 chaperone protein or modulates the interaction of Hsp90 with a client protein, e.g., by targeting, decreasing or inhibiting the intrinsic ATPase activity of Hsp90.

In one embodiment the interaction modulated by a Hsp90 inhibitor is the interaction between Hsp90 and tyrosine hydroxylase. In one embodiment the Hsp90 inhibitor disturbs or disrupts the interaction of Hsp90 with tyrosine hydroxylase.

The term “Hsp90 modulator” as used herein refers to a compound (1) that induces one or more posttranslational modifications on Hsp90 via proteins, such as histonedeacetylases (HDACs); or (2) that inhibits the formation of the Hsp90 complex; leading to disruption of structure and/or function of an Hsp90 chaperone protein or the modulation of the interaction of Hsp90 with a client protein. For example, Hsp90 can be acetylated and inactivated by histone deacetylase (HDAC) inhibitors (Kovacs et al., Mol. Cell. 2005, 18, 601-607; Fuino et al., Mol. Cancer. Ther. 2003, 2, 971-984; Aoyagi et al., Trends Cell. Biol. 2005, 15, 565-567).

In one embodiment the interaction modulated by a Hsp90 modulator is the interaction between Hsp90 and typrosine hydroxylase. In one embodiment the Hsp90 modulator reduces, disturbs or disrupts the interaction between Hsp90 and tyrosine hydroxylase.

The term “Hsp90 complex” as used herein refers to an aggregate of on or more proteins, including Hsp90 and other co-chaperones such as Hsp70, Hsp27, Hsp40, HOP, p23, and CDC37.

The term “tyrosine hydroxylase modulator” or “TH modulator” as used herein refers to a compound, such as a small molecule, that interacts with tyrosine hydroxylase capable of modulating the interaction of tyrosine hydroxylase with Hsp90 or the Hsp90 complex. In one embodiment the TH modulator reduces or disturbs the interaction of Hsp90 or the Hsp90 complex with tyrosine hydroxylase. In a further embodiment the TH modulator disrupts the interaction of Hsp90 or the Hsp90 complex with tyrosine hydroxylase. In one embodiment the TH modulator is L-alpha-methyl-p-tyrosine (metirosine, Demser; see: Voorhess, Cancer Research 1968, 28, 452).

The term “modulators that reduce the interaction between Hsp90 and tyrosine hydroxylase” or “Hsp90-TH modulator” as used herein refers to a compound, such as a small molecule, that interacts with the Hsp90 tyrosine hydroxylase complex. In one embodiment the Hsp90-TH modulator intercalates between the Hsp90-TH complex and reduces the interaction. In another embodiment the Hsp90-TH modulator interacts with a binding site resulting from, for example, the conformational change of Hsp90 or TH within the Hsp90-TH complex. In another embodiment the Hsp90-TH modulator interacts with a novel catalytic site or binding site within the Hsp90-TH complex. In another embodiment the Hsp90-TH modulator induces a conformational change within the Hsp90-TH complex dissociating or reducing the interaction within the Hsp90-TH complex.

As used herein, the term “small molecules” refers to small organic or inorganic molecules of molecular weight below 5,000 Daltons. In one embodiment small molecules useful for the invention have a molecular weight of less than 1,000 Daltons. In one embodiment small molecules useful for the invention have a molecular weight of less than 500 Daltons.

As used herein, the term “exemplified compound” comprises all compounds pertaining to a general formula disclosed in a publication that have been generated and isolated, e.g., by chemical synthesis or biotechnological methods. For example Snader et al., WO02/079167 discloses general formula I (p. 2). The compounds that have been obtained, compound A, B, and C1 thus constitute the “exemplified compounds of formula I” of this publication (pp. 11-12).

The term “alkyl” refers to a linear or branched saturated monovalent hydrocarbon radical. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆₋) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl may be substituted.

The term “alkylene” refers to a linear or branched saturated divalent hydrocarbon radical, wherein the alkylene may optionally be substituted. The term “alkylene” encompasses both linear and branched alkylene, unless otherwise specified. In certain embodiments, the alkylene is a linear saturated divalent hydrocarbon radical that has 1 to 20 (C₁₋₂₀), 1 to 15 (C₁₋₁₅), 1 to 10 (C₃₋₁₀), or 1 to 6 (C₃₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkylene groups are also referred as “lower alkylene.” Examples of alkylene groups include, but are not limited to, methylene, ethylene, propylene (including all isomeric forms), n-propylene, isopropylene, butylene (including all isomeric forms), n-butylene, isobutylene, t-butylene, pentylene (including all isomeric forms), and hexylene (including all isomeric forms). For example, C₂₋₆ alkylene refers to a linear saturated divalent hydrocarbon radical of 2 to 6 carbon atoms or a branched saturated divalent hydrocarbon radical of 3 to 6 carbon atoms.

The term “alkenyl” refers to a linear or branched monovalent hydrocarbon radical, which contains one or more carbon-carbon double bonds. The alkenyl may be optionally substituted, e.g., as described herein. The term “alkenyl” also embraces radicals having “cis” and “trans” configurations, or alternatively, “E” and “Z” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₂₀), or 2 to 6 (C₂₋₆) carbon atoms or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl, allyl, propenyl, butenyl, and 4-methylbutenyl.

The term “alkenylene” refers to a linear or branched divalent hydrocarbon radical, which contains one or more carbon-carbon double bonds. The alkenylene may be optionally substituted, e.g., as described herein. Similarly, the term “alkenylene” also embraces radicals having “cis” and “trans” configurations, or alternatively, “E” and “Z” configurations. As used herein, the term “alkenylene” encompasses both linear and branched alkenylene, unless otherwise specified. For example, C₂₋₆ alkenylene refers to a linear unsaturated divalent hydrocarbon radical of 2 to 6 carbon atoms or a branched unsaturated divalent hydrocarbon radical of 3 to 6 carbon atoms. In certain embodiments, the alkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenylene groups include, but are not limited to, ethenylene, propenylene, allylene, propenylene, butenylene, and 4-methylbutenylene.

The term “alkynyl” refers to a linear or branched monovalent hydrocarbon radical, which contains one or more carbon-carbon triple bonds. The alkynyl may be optionally substituted, e.g., as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “alkynylene” refers to a linear or branched divalent hydrocarbon radical, which contains one or more carbon-carbon triple bonds. The alkynylene may be optionally substituted, e.g., as described herein. The term “alkynylene” also encompasses both linear and branched alkynylene, unless otherwise specified. In certain embodiments, the alkynylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynylene groups include, but are not limited to, ethynylene (—C≡C—) and propargylene (—CH₂C≡C—). For example, C₂₋₆ alkynyl refers to a linear unsaturated divalent hydrocarbon radical of 2 to 6 carbon atoms or a branched unsaturated divalent hydrocarbon radical of 3 to 6 carbon atoms.

The term “cycloalkyl” refers to a cyclic saturated bridged or non-bridged monovalent hydrocarbon radical, which may be optionally substituted, e.g., as described herein. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, decalinyl, and adamantyl.

The term “cycloalkylene” refers to a cyclic saturated bridged or non-bridged divalent hydrocarbon radical, which may be optionally substituted, e.g., as described herein. In certain embodiments, the cycloalkylene has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkylene groups include, but are not limited to, cyclopropylene, cyclobutylene, cyclopentylene, cyclohexylene, cycloheptylene, decalinylene, and adamantylene. The term “aryl” refers to a monocyclic or multicyclic monovalent aromatic group. In certain embodiments, the aryl has from 6 to 20 (C₆₋₂₀), from 6 to 15 (C₃₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). All such aryl groups may also be optionally substituted, e.g., as described herein.

The term “arylene” refers to a monocyclic or multicyclic divalent aromatic group. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene, naphthylene, fluorenylene, azulenylene, anthrylene, phenanthrylene, pyrenylene, biphenylene, and terphenylene. Arylene also refers to bicyclic or tricyclic carbon rings, where one of the rings is aromatic and the others of which may be saturated, partially unsaturated, or aromatic, for example, dihydronaphthylene, indenylene, indanylene, or tetrahydro-naphthylene (tetralinyl). All such aryl groups may also be optionally substituted, e.g., as described herein.

The term “heteroaryl” refers to a monocyclic or multicyclic aromatic group, wherein at least one ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. All such heteroaryl groups may also be optionally substituted, e.g., as described herein.

The term “heterocyclyl” or “heterocyclic” refers to a monocyclic or multicyclic non-aromatic ring system, wherein one or more of the ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. Examples of heterocyclyl groups include, but are not limited to, pyrrolidinyl, piperidinyl, 2-oxopyrrolidinyl, 2-oxopiperidinyl, morpholinyl, piperazinyl, tetrahydropyranyl, and thiomorpholinyl. All such heterocyclic groups may also be optionally substituted, e.g., as described herein.

The term “alkoxy” refers to an —OR radical, wherein R is, for example, alkyl, alkenyl, alkynyl, cycloalkyl, aryl, heteroaryl, or heterocyclyl, each as defined herein. Examples of alkoxy groups include, but are not limited to, methoxy, ethoxy, propoxy, n-propoxy, 2-propoxy, n-butoxy, isobutoxy, tert-butoxy, cyclohexyloxy, phenoxy, benzoxy, and 2-naphthyloxy.

The term “acyl” refers to a —C(O)R radical, wherein R is, for example, alkyl, alkenyl, alkynyl, cycloalkyl, aryl, heteroaryl, or heterocyclyl, each as defined herein. Examples of acyl groups include, but are not limited to, acetyl, propionyl, butanoyl, isobutanoyl, pentanoyl, hexanoyl, heptanoyl, octanoyl, nonanoyl, decanoyl, dodecanoyl, tetradecanoyl, hexadecanoyl, octadecanoyl, eicosanoyl, docosanoyl, myristoleoyl, palmitoleoyl, oleoyl, linoleoyl, arachidonoyl, benzoyl, pyridinylcarbonyl, and furoyl.

The term “halogen”, “halide” or “halo” refers to fluorine, chlorine, bromine, or iodine.

The term “optionally substituted” is intended to mean that a group, such as an alkyl, alkylene, alkenyl, alkenylene, alkynyl, alkynylene, cycloalkyl, cycloalkylene, aryl, arylene, heteroaryl, or heterocyclyl group, may be substituted with one or more substituents independently selected from, e.g., halo, cyano (—CN), nitro (—NO₂), —SR, —S(O)R^{a}, S(O)₂R^{a}, —R^{a}, C(O)R^{a}, —C(O)OR^{a}, —C(O)NR^{b}R^{c}, —C(NR^{a})NR^{b}R^{c}, —OR^{a}, —OC(O)OR^{a}, —OC(O)NR^{b}R^{c}, —OC(═NR^{a})NR^{b}R^{c}, —OS(O)R^{a}, OS(O)₂R^{a}, —OS(O)NR^{b}R^{c}, —OS(O)₂NR^{b}R^{c}, —NR^{b}R^{c}, NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)R^{b}R^{c}, or —NR^{a}S(O)₂R^{b}R^{c}; wherein R^{a}, R^{b}, R^{c}, and R^{d} are each independently, e.g., hydrogen, alkyl, alkenyl, alkynyl, cycloalkyl, aryl, heteroaryl, or heterocyclyl, each optionally substituted, e.g., as described herein; or R^{b} and R^{c} together with the N atom to which they are attached form heterocyclyl or heteroaryl, each optionally substituted, e.g., as described herein. The group can be substituted with any described moiety, including, but not limited to, one or more moieties selected from the group consisting of halogen (fluoro, chloro, bromo, or iodo), hydroxyl, amino, alkylamino (e.g., monoalkylamino, dialkylamino, or trialkylamino), arylamino (e.g., monoarylamino, diarylamino, or triarylamino), alkoxy, aryloxy, nitro, cyano, sulfonic acid, sulfate, phosphonic acid, phosphate, or phosphonate, either unprotected or protected as necessary, as known to those skilled in the art, for example, as taught in Greene, et al., Protective Groups in Organic Synthesis, John Wiley and Sons, Second Edition, 1991. As used herein, all groups that can be substituted in one embodiment are “optionally substituted,” unless otherwise specified.

### 7.2 Hsp90 Inhibitors, Hsp90 Modulators, Tyrosine Hydroxylase Modulators and Hsp90-TH Modulators

The eukaryotic heat shock protein 90s (Hsp90s) are ubiquitous chaperone proteins that bind and hydrolyze ATP. Hsp90s are believed to be involved in folding, activation and assembly of a number of client proteins, including proteins involved in signal transduction, cell cycle control, and transcriptional regulation.

Hsp90 proteins are highly conserved in nature, and include Hsp90 alpha and beta, Grp94, and Trap-1. For exemplary protein sequences see, e.g., NCBI accession Nos. NP_{—}005339.2 and NP_{—}001014390.1 (Homo sapiens alpha and beta Hsp90, respectively); P07901 (Mus musculus); NP_{—}001004082.2, AAT99568.1 (Rattus norvegicus); AAA36992.1 (Cricetulus griseus); JC1468 and HHCH90 (Gallus gallus); AAF69019.1 (Sarcophaga crassipalpis); AAC21566.1 (Danio rerio), AAD30275.1 (Salmo salar), NP_{—}999138.1 (Sus scrofa), NP₁₃ 015084.1 (Saccharomyces cerevisiae), and CAC29071 (frog).

The Hsp90 inhibitors or modulators can be specifically directed against an Hsp90 of the specific host patient, or can be identified based on reactivity against an Hsp90 homolog from a different species, or an Hsp90 variant.

In vivo and in vitro studies indicate that without the aid of co-chaperones Hsp90 is unable to fold or activate proteins. For steroid receptor conformation and association in vitro, Hsp90 requires Hsp70 and p60/Hop/Sti1 (Caplan et al., Trends in Cell Biol. 1999, 9, 262-268). In vivo Hsp90 may interact with Hsp70 and its co-chaperones. Other co-chaperones associated with Hsp90s in higher eukaryotes include Hip, Bag1, Hsp40/Hdj2/Hsj1, immunophilins, p23, and p50 (Caplan et al., 1999 supra). The binding of ansamycins to Hsp90 has been reported to inhibit protein refolding and to cause the proteasome dependent degradation of a select group of cellular proteins (Sepp-Lorenzino et al., J. Biol. Chem. 1995, 270, 16580-16587; Whitesell et al., Proc. Natl. Acad. Sci. USA 1994, 91, 8324-8328).

Tyrosine hydroxylase (TH) catalyzes the hydroxylation of L-tyrosine to L-3,4-dihydroxyphenylalanine, which is the rate limiting step in the biosynthesis of catecholamine neurotransmitters, including dopamine (Nagatsu et al., J. Biol. Chem. 1964, 239, 2910-2917; Levitt et al., J. Pharmacol. Exp. Ther. 1965, 148, 1-8). The mesolimbic dopamine system, which consists of the dopaminergic neurons in the ventral tegmental area (VTA) and projections to the nucleus accumbens and the prefrontal cortex, is the major neural structure that mediates the rewarding effects of drugs of abuse and ethanol. Biochemical adaptations in dopaminergic midbrain neurons induced by chronic exposure to drugs of abuse have been observed and implicated in relation to drug addiction (Self et al., Annu. Rev. Neurosci. 1995, 18, 463-495; Nestler et al., J. Neurosci. 1992, 12, 2439-2450; Koob et al., Trends Pharmacol. Sci. 1992, 13, 177-184; Wise et al., Annu. Rev. Psychol. 1989, 40, 191-225). One of the most consistent adaptations to long-term exposure to drugs of abuse, including ethanol, is the upregulation of TH protein levels in the VTA. Upregulation of TH levels has been reported as a hallmark of biochemical adaptations to in vivo chronic exposure to drugs of abuse, including ethanol. (Messer et al., Neuron 2000, 26, 247-257; Ortiz et al., Synapse 1995, 21, 289-298; Beitner-Johnson et al., J. Neurochem. 1991, 57, 344-347; Sorg et al., J. Pharmacol. Exp. Ther. 1993, 266, 424-430; Haile et al., Synapse 2001, 41, 179-190; Schmidt et al., J. Neurosci. 2001, 21, RC137, 1-5).

It was now found that prolonged exposure of the dopaminergic like SH-SY5Y cells to ethanol produces a similar adaptation in TH protein levels as previously reported in vivo (Ortiz et al., supra). This prolonged exposure induces a long-lasting increase in TH protein levels by increasing the association of TH with the chaperone protein Hsp90 via the cAMP/PKA pathway, leading to further enhancement of protein stability.

It was also found that chronic ethanol treatment does not induce TH gene transcription or translation. Instead the data presented in the instant disclosure demonstrates that an increase in the maintenance of TH protein stability leads to the accumulation of the protein in response to ethanol exposure.

A novel mechanism via Hsp90 by which TH protein is stabilized following the exposure of the dopaminergic-like cells to ethanol is presented herein. Most importantly it was discovered that Hsp90 inhibitors or modulators, such as Geldanamycin, dose-dependently inhibit ethanol-mediated increases in TH protein.

Small Molecules

In one embodiment the Hsp90 inhibitors or modulators include small molecules chosen from, but not limited to, purine scaffold-based compounds (See, for example, Chiosis et al., WO 02/036075; Kasibhatla et al., WO 03/037860; Chiosis et al., WO 06/084030; and Chiosis et al., WO/2008/005937); pyrazole or imidazole scaffold-based compounds (see, for example, Ying et al., WO 07/021,877; Barill et al., Bioorg Med. Chem. Lett. 2006, 16, 2543-2548, or Sharp et al., Molecular Cancer Therapeutics 2007, 6, 1198-1211); tetrahydroindolone or tetrahydroindazolone derivatives (See Huang et al., WO 06/091963); or hydroxamic acids (See Schaefer et al., Bioorg. Med. Chem. Nov. 4, 2007 (article in press); Elaut et al., Curr. Pharm. Des. 2007, 13, 2584-2620; Balakin et al., Anticancer Agents Med Chem. 2007, 7, 576-92; Secrist et al., Curr. Opin. Investig. Drugs 2003, 4, 1422-1427).

In one embodiment the Hsp90 inhibitor or modulator is the amino-glycoside antibiotic, Novobiocin (see Yu et al., J. Am. Chem. Soc. 2005, 127, 12778-12779).

In one embodiment the Hsp90 inhibitor or modulator is radicicol or an analog thereof, such as oxime, ester and palmitoyl derivatives (see Agatsuma et al., WO 96/033989; Ino et al., WO 98/18780; Ino et al., WO 1999/55689; Danishefsky et al., U.S. Pat. No. 7,115,651; Feng et al., U.S. Pat. No. 5,731,343; Kato et al., U.S. Pat. No. 5,077,165; Soga et al., Current Cancer Drug Targets 2003, 3, 359-369; Ki et al., J. Biol. Chem. 2000, 275, 39231-39236).

Ansamycin Derivatives

In one embodiment, suitable Hsp90 inhibitors include compounds that bind to the ATP/ADP-binding pocket in the geldanamycin-binding domain of Hsp90 (residues 9-232 of human Hsp90, see Stebbins et al., Cell 1997, 89, 239-250; Schulte et al., Cell Stress Chaperones 1998, 3, 100-108).

Geldanamycin Derivatives

In one embodiment the ansamycin derivatives include the benzoquinone ansamycins. Examples of such compounds include, but are not limited to, geldanamycin and geldanamycin derivatives, such as 17-alkylamino-17-desmethoxy-geldanamycin (“17-AAG”) and 17-(2 dimethylaminoethyl)amino-17-desmethoxy-geldanamycin (“17-DMAG”)). See Sasaki et al., U.S. Pat. No. 4,261,989 for synthesis of 17-AAG and Snader et al., US 2004/0053909 for synthesis of 17-DMAG.

In one embodiment the Hsp90 inhibitor is geldanamycin.

In one embodiment the Hsp90 inhibitor is 17-AAG.

In one embodiment the Hsp90 inhibitor is 17-DMAG.

In one embodiment the Hsp90 inhibitor is a compound of formulae II or III:

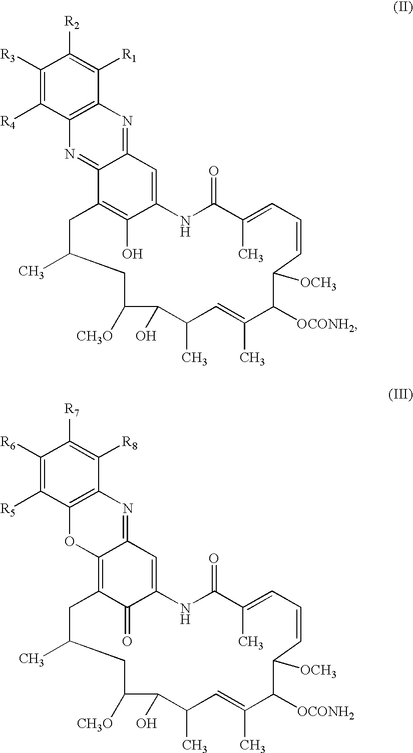

wherein

R₁, R₂, R₃, and R₄ are the same or different and selected from hydrogen, hydroxy, halo, alkyl, alkoxyl, carboxyl, carboalkoxyl, amino, carbamido, carboxamido or N-substituted carboxamido; and

R₅, R₆, R₇ and R₈ are the same or different and selected from hydrogen, hydroxy, halo, alkyl, alkoxyl, carboxyl, carboalkoxyl, amino, amido, or N-alkyl substituted amido.

In one embodiment the substituents of formulae II and III are defined as in Rinehart et al., U.S. Pat. No. 3,987,035. The compounds of formulae II and III may be synthesized as described in Rinehart et al., U.S. Pat. No. 3,987,035.

In a further embodiment the Hsp90 inhibitor is selected from a group comprising the exemplified compounds of formulae II and III as disclosed in Rinehart et al., U.S. Pat. No. 3,987,035.

In one embodiment the Hsp90 inhibitor is a compound of formula IV:

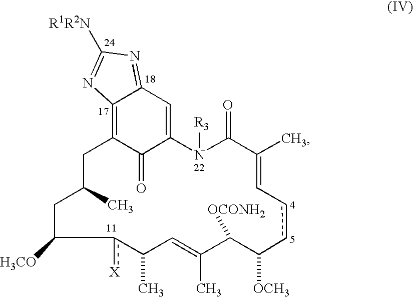

wherein the dotted lines between the carbon atoms at positions “4” and “5” and between X and the carbon at position “11” represent optional double bonds;

and wherein R¹ and R² are independently selected from hydrogen, (C₁-C₈)alkyl, (C₁-C₈)alkenyl, (C₁-C₈)alkynyl, (C₃-C₇)cycloalkyl and phenyl-(C₁-C₃)alkyl, wherein the phenyl moiety of said phenyl-(C₁-C₃)alkyl may optionally be substituted with from one to three substituents independently selected from halo, azido, nitro, (C₁-C₆)alkyl, (C₁-C₆)alkoxy, aryl, cyano and NR⁴R⁵R⁶, wherein R⁴, R⁵ and R⁶ are independently selected from hydrogen and (C₁-C₆)alkyl;

or R¹ and R² can form, together with the nitrogen to which they are attached, a heterocyclic ring selected from aziridine, azetidine, pyrrolidine, thiazolidine, oxazolidine, piperidine, morpholine, piperazine, 4-(C₁-C₄)alkylpiperidine, N—(C₁-C₆)alkylpiperazine and N-benzylpiperazine;

R³ is hydrogen or a group of the formula

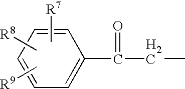

wherein R⁷, R⁸ and R⁹ are independently selected from hydrogen, halo, azido, nitro, (C₁-C₈)alkyl, (C₁-C₈)alkoxy, aryl, cyano and NR¹⁰R¹¹R¹² wherein R¹⁰, R¹¹ and R¹² are independently selected from hydrogen and (C₁-C₃)alkyl;

X is halo or OR¹³ when there is a single bond between X and the carbon at position “11”, and X is oxo (═O) or oximino (═NOH) when there is a double bond between X and the carbon at position “11”;

R¹³ is selected from the group consisting of hydrogen, R¹⁴C(=0), R¹⁴SO₂ and R¹⁵R¹⁶NSO₂NHC(═O);

R¹⁴ is selected from the group consisting of hydrogen, (C₁-C₈)alkyl, amino(C₁-C₈) alkyl, hydroxy(C₁-C₈)alkyl and aryl, wherein said aryl is selected from phenyl and naphthyl, and wherein said aryl, (C₁-C₈)alkyl and the alkyl moieties of said amino(C₁-C₈) alkyl and hydroxy(C₁-C₈)alkyl may be substituted with one or more substituents, preferably with from zero to three substituents, independently selected from (C₁-C₈)alkyl, halo, amino, nitro, azido, hydroxy and (C₁-C₈)alkoxy; and

R¹⁵ and R¹⁸ are independently selected from hydrogen, (C₁-C₈)alkyl, (C₁-C₈)alkenyl, (C₃-C₇)cycloalkyl, amino-(C₁-C₈)alkyl, hydroxy-(C₁-C₈)alkyl and methoxy-(C₁-C₈)alkyl;

or R¹⁵ and R¹⁶ form, together with the nitrogen to which they are attached, a heterocyclic ring selected from aziridine, azetidine, pyrrolidine, thiazolidine, oxazolidine, piperidine, morpholine, piperazine, 4-(C₁-C₄)alkylpiperidine, N(C₁-C₆)alkylpiperazine and N-benzylpiperazine.

Compounds of the formula I wherein IV is single bonded to the carbon at position “11” contain a chiral center at position “11”. This invention relates to all stereoisomers of compounds of the formula I, including racemic mixtures thereof, that derive from the chirality of the carbon at position “11”.

Preferred compounds of the formula IV include those wherein: (a) each of R¹ and R² is methyl and X is hydroxy; (b) R¹ is methyl, R² is benzyl and X is hydroxy, or (c) R¹ and R², together with the nitrogen to which they are attached, form a 4-methylpiperidine ring, and X is hydroxy.

In one embodiment the substituents of formula IV are defined as in Schnur, WO94/22867. The compounds of formula IV may be synthesized as described in Schnur, WO94/22867.

In a further embodiment the Hsp90 inhibitor is selected from a group comprising the exemplified compounds of formula IV as disclosed in Schnur, WO94/22867.

In one embodiment the Hsp90 inhibitor is a compound of formula V:

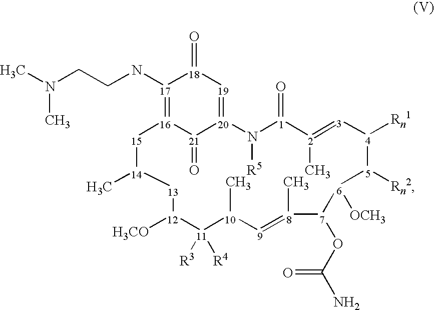

wherein n=0 or 1 and wherein, when n=1, each of R¹ and R² is hydrogen, and, when n=0, a double-bond exists between C₄ and C₅;

R³ is hydrogen or hydroxyl;

R⁴ is hydrogen or hydroxyl; wherein, when R³ is hydrogen, R⁴ is hydroxyl, and when R³ is hydroxyl, R⁴ is hydrogen; and

R⁵ is hydrogen or a group of the formula

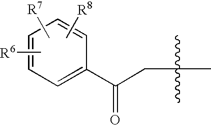

wherein each of R⁶, R⁷, and R⁸ is independently selected from the group consisting of hydrogen, halo, azido, nitro, a C₁-C₈ alkyl, a C₁-C₈ alkoxy, aryl, cyano, and NR¹⁰R¹¹R¹², wherein each of R¹⁰, R¹¹, and R¹² is independently selected from the group consisting of hydrogen and a C₁-C₃ alkyl; and salts thereof.

In one embodiment the substituents of formula V are defined as in Snader, WO02/079167. The compounds of formula V may be synthesized as described in Snader, WO02/079167.

In a further embodiment the Hsp90 inhibitor is selected from a group comprising the exemplified compounds of formula V as detailed in Snader, WO02/079167.

In one embodiment the Hsp90 inhibitor is a compound of formula VI:

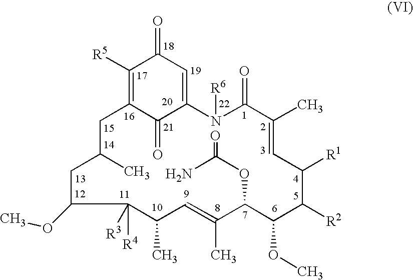

wherein R¹ and R² are both hydrogen or R¹ and R² together form a single bond;
wherein R¹ and R² are both hydrogen or R¹ and R² together form a single bond;

R³ is hydrogen and R⁴ is selected from the group consisting of —OR¹⁰, —NHR⁸ and halo;

wherein R¹⁰ is selected from the group consisting of hydrogen, R¹¹C(═O)—, RSO₂— and R¹²R¹³NSO₂NHC(═O)—;
wherein R¹¹ is selected from the group consisting of amino, (C₁-C₈)alkyl, amino(C₁-C₈)alkyl, hydroxy(C₁-C₈)alkyl, protected amino(C₁-C₈)alkyl, protected hydroxy(C₁-C₈)alkyl, phenyl and naphthyl; and
R¹² and R¹³ are each independently selected from the group consisting of hydrogen, (C₁-C₈)alkyl, amino (C₁-C₈)alkyl, dimethylamino (C₁-C₈)alkyl, cyclo(C₃-C₈)alkyl, phenyl and naphthyl; or R¹² and R¹³ together with the nitrogen to which they are attached form a heterocyclic residue selected from the group consisting of aziridinyl, azetidinyl, pyrrolidinyl, piperidinyl, thiazolidinyl, oxazolidinyl, morpholino, piperazinyl, 4-(C₁-C₄)alkylpiperidinyl and N—(C₁-C₄)piperazinyl;
and said alkyl, phenyl and naphthyl groups may be substituted with one or more residues selected from the group consisting of (C₁-C₈)alkyl, halo, nitro, amino, azido and (C₁-C₈)alkoxyl; or

R³ and R⁴ together form a group of the formula

=J

wherein J is selected from O and NOH;

R⁵ is NR⁸R⁹ wherein R⁸ and R⁹ are each independently selected from the group consisting of hydrogen, (C₁-C₈)alkyl, (C₃-C₈)cycloalkyl, (C₂-C₈)alkenyl and (C₂-C₈)alkynyl; wherein said alkyl, alkenyl and alkynyl are optionally substituted wherein said substituents are selected from the group consisting of halo, cyano, mercapto, (C₁-C₈)alkylthio, optionally substituted amino, hydroxyl, (C₁-C₈)alkoxyl, carboxyl, amidino, acylamino, and (C₂-C₆)heterocycloalkyl and (C₂-C₆)heterocycloaryl groups selected from the group comprising imidizaloly, furyl, tetrahydrofuryl; and if comprising more than two carbon atoms may be branched, cyclic or unbranched or combinations of branched, cyclic and unbranched groups; or R⁸ and R⁹ together with the nitrogen to which they are attached form a heterocyclic residue selected from the group consisting of aziridinyl, azetidinyl and pyrrolidinyl;

or R⁵ is R¹⁴ O wherein R¹⁴ is hydrogen or (C₁-C₄)alkyl;

and R⁶ is hydrogen or a group of the formula

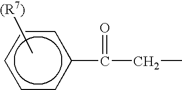

wherein m is 0 or an integer from 1-5 and each R⁷ is independently selected from halo, azido, nitro, (C₁-C₈)alkyl, (C₁-C₈)alkoxyl, phenyl and naphthyl, cyano and NR⁸R⁹ wherein R⁸ and R⁹ are as defined above; with the proviso that when R¹ and R² together form a single bond R³ is hydrogen and R⁴ is OR¹⁰ wherein R¹⁰ is hydrogen then R⁵ cannot be OR¹⁴, wherein R¹⁴ is hydrogen or methyl, or NR⁸R⁹ wherein HNR⁸R⁹ is selected from the group consisting of ammonia, methylamine, ethylamine, propylamine, butylamine, pentylamine, hexylamine, heptylamine, octylamine, allylamine, β-hydroxyethylamine, β-chloroethylamine, β-glycoxyethylamine, aminobutylamine, adamantylmethylamine, cyclopropylamine, cyclopentylamine, cyclohexylamine, cycloheptylamine, cyclooctylamine, benzylamine, phenethylamine, ethyleneamine, pyrrolidine, piperidine, dimethylamine, aminoethylamine, diglycolamine, β-morpholinoethylamine, β-piperidinoethylamine, picolylamine, β-pyrrolidinoethylamine, β-pyridinylethylamine, β-methoxyethylamine, and β-N-methylaminoethylamine; and when R⁵ is OR¹⁴ and R¹⁰ is R¹¹C(═O), R¹¹ cannot be methyl.

In one embodiment the substituents of formula VI are defined as in Gallaschun et al., WO95/01342. The compounds of formula VI may be synthesized as described in Gallaschun et al., WO95/01342.

In a further embodiment the Hsp90 inhibitor is selected from a group comprising the exemplified compounds of formula VI as detailed in Gallaschun et al., WO95/01342.

In one embodiment the Hsp90 inhibitor is a compound of formula VII:

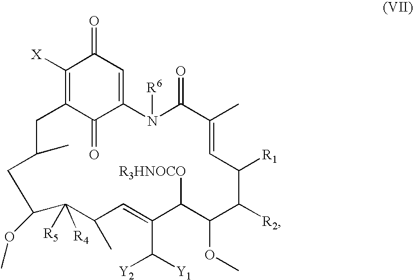

wherein X is selected from the group consisting of optionally substituted (C₁-C₂₀) alkyl, optionally substituted (C₁-C₂₀)heteroalkyl, optionally substituted (C₂-C₂₀)alkenyl, optionally substituted (C₂-C₂₀)heteroalkenyl, optionally substituted (C₂-C₂₀)alkynyl, optionally substituted aryl, optionally substituted heteroaryl, optionally substituted arylalkyl, optionally substituted heteroarylalkyl, optionally substituted cycloalkyl, optionally substituted cycloheteroalkyl, —N(R₉)—C(O)R₇, —N(R₉)—C(O)—OR₇, —N(R₉)—C(O)—NR₇R₈, —N(R₉)—C(S)R₇, —N(R₉)—C(S)—OR₇, —N(R₉)—C(S)—NR₇, R₈, —OR₆ and —N(R₁₄)(R₁₅);

R₁ and R₂ are both H or together form a bond;

R₃ is selected from the group consisting of H and optionally substituted C₁-C₃ alkyl

R₄ and R₅ are independently selected from the group consisting of H, —OH, O— alkyl, O-acetyl, —O-aryl, OC(O)R₁₀, —SO₂—R₁₀, and —NHR₁₀, or together form oxo (═O), or hydroxylamino alkoxyimine or aryloxyimine, thioketo, wherein R₁₀ is selected from the group consisting of H, optionally substituted (C₁-C₂₀) alkyl, optionally substituted (C₁-C₂₀) heteroalkyl, optionally substituted aryl; and optionally substituted heteroaryl;

R₆ is selected from the group consisting of H, optionally substituted C₁-C₈ alkyl, optionally substituted C₅-C₈ aryl, and optionally substituted C₁-C₆ acyl;

R₇ and R₈ each independently is selected from the group consisting of H, optionally substituted (C₁-C₂₀)alkyl, optionally substituted (C₁-C₂₀)heteroalkyl, optionally substituted (C₂-C₂₀)alkenyl, optionally substituted (C₂-C₂₀)heteroalkenyl, optionally substituted (C₂-C₂₀)alkynyl, optionally substituted aryl, optionally substituted heteroaryl, optionally substituted arylalkyl, optionally substituted heteroarylalkyl, optionally substituted cycloalkyl, and optionally substituted cycloheteroalkyl; or together form a 4-7 membered optionally substituted ring;

R₉ is selected from the group consisting of H, optionally substituted C₁-C₆ alkyl, optionally substituted C₅-C₈ aryl, and optionally substituted C₅-C₈ heteroaryl, or together with R₇ or R₈ forms a 4-7 membered optionally substituted ring;

R₁₄ and R₁₅ are independently selected from the group consisting of H, optionally substituted (C₁-C₂₀)alkyl, optionally substituted (C₂-C₂₀)heteroalkyl optionally substituted (C₂-C₂₀)alkenyl, optionally substituted (C₂-C₂₀)heteroalkenyl, optionally substituted (C₂-C₂₀)alkynyl, optionally substituted aryl, optionally substituted heteroaryl, optionally substituted arylalkyl, optionally substituted heteroarylalkyl, optionally substituted cycloalkyl, and optionally substituted cycloheteroalkyl; or R₁₄ and R₁₅ together form an optionally substituted 4-7 membered heterocyclic or carbocyclic ring;

wherein Y₁ and Y₂ are independently selected from the group consisting H, —OH, O-alkyl, O-acetyl, —O-aryl, OC(O)R₁₀, —SO₂—R₁₀, and —NHR₁₀, or together form oxo (═O), or hydroxylamino alkoxyimine or aryloxyimine, thioketo, wherein R₁₀ is selected from H, optionally substituted (C₁-C₂₀)alkyl, optionally substituted (C₁-C₂₀)heteroalkyl, optionally substituted aryl and optionally substituted heteroaryl; or

Y₁, or Y₂ taken with R₄ or R₅ form an optionally substituted 5-7 membered heterocyclic or carbocyclic ring;

provided that, if Y₁ and Y₂ are both H and X is N(R₁₄) (R₁₅) or —OR₆, then at least one of R₄, R₅, R₆, R₁₄, and R₁₅ comprises a phosphorous moiety selected from the group consisting of —OP(O)(OR₁₆)₂ (phosphate), —CH₂P(O)(OR₁₆)₂ (phosphonate), and —NP(O)(OR₁₆)₂ (phosphoramide), wherein R₁₆ is selected from the group consisting of H, alkyl, heteroalkyl, alkenyl, heteroalkenyl, alkynyl, heteroalkynyl, aryl and heteroaryl.

In one embodiment the substituents of formula VII are defined as in Zhang et al., WO03/066005. The compounds of formula VII may be synthesized as described in Zhang et al., WO03/066005.

In a further embodiment the Hsp90 inhibitor is selected from a group comprising the exemplified compounds of formula VII as detailed in Zhang et al., WO03/066005.

In one embodiment the Hsp90 inhibitor is a compound of formula VIII:

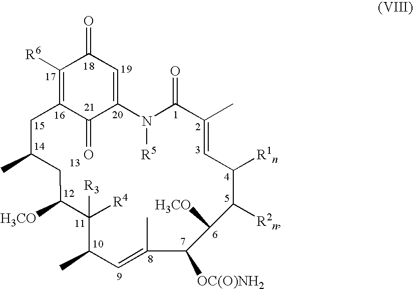

wherein n=0 or 1 and wherein, when n=1, each of R¹ and R² is hydrogen, and, when n=0, a double-bond exists between position 4 and position 5;

R³ is hydrogen or hydroxyl;

R⁴ is hydrogen, hydroxyl, or R⁷C(O)O—, wherein R⁷ is amino (C₁-C₈)alkyl or imino (C₁-C₈)alkyl; wherein, when R³ is hydrogen, R⁴ is hydroxyl or R⁷C(O)O—, and when R³ is hydroxyl, R⁴ is hydrogen;

R⁵ is hydrogen or a group of the formula

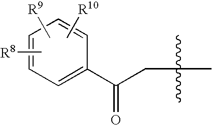

wherein each of R⁸, R⁹, and R¹⁰ is independently selected from the group consisting of hydrogen, a halo, an azido, a nitro, a C₁-C₈ alkyl, a C₁-C₈ alkoxy, an aryl, a cyano, and an NR¹¹R¹²R¹³, wherein each of R¹¹, R¹², and R¹³ is independently selected from the group consisting of hydrogen and a C₁-C₃alkyl;

R⁶ is hydrogen, a methoxy, a C₁-C₈ alkylamino, a C₁-C₈ dialkylamino, an N,N′-dialkylaminodialkylamino-, an N,N′-dialkylaminoalkylamino, or an allylamino.

In one embodiment the substituents of formula VIII are defined as in Fumo et al., WO04/037978. The compounds of formula VIII may be synthesized as described in Fumo et al., WO04/037978.

In a further embodiment the Hsp90 inhibitor is selected from a group comprising the exemplified compounds of formula VIII as detailed in Fumo et al., WO04/037978.

In one embodiment the Hsp90 inhibitor is a compound of formula IX:

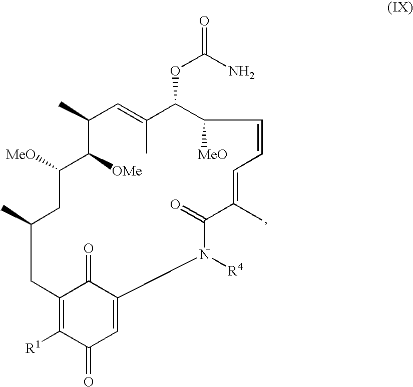

wherein

R¹ is OMe or R²R³N, where R² and R³ are independently H, C₁-C₈ alkyl, C₂ C₈ alkenyl, C₂-C₈ alkynyl, cycloalkyl, heterocyclo, aryl, or heteroaryl; or R² and R³ and the nitrogen to which they are attached combine to form a substituted or unsubstituted 3, 4, 5, 6, or 7 membered ring; and

R⁴ is H or CH₂C(═O)R⁵, where R⁵ is a substituted or unsubstituted phenyl group.

In one embodiment the substituents of formula IX are defined as in Tian et al., U.S. Pat. No. 6,855,705. The compounds of formula IX may be synthesized as described in Tian et al., U.S. Pat. No. 6,855,705.

In a further embodiment the Hsp90 inhibitor is selected from a group comprising the exemplified compounds of formula IX as detailed in Tian et al., U.S. Pat. No. 6,855,705.

In one embodiment the Hsp90 inhibitor is a compound of formulae X and XI:

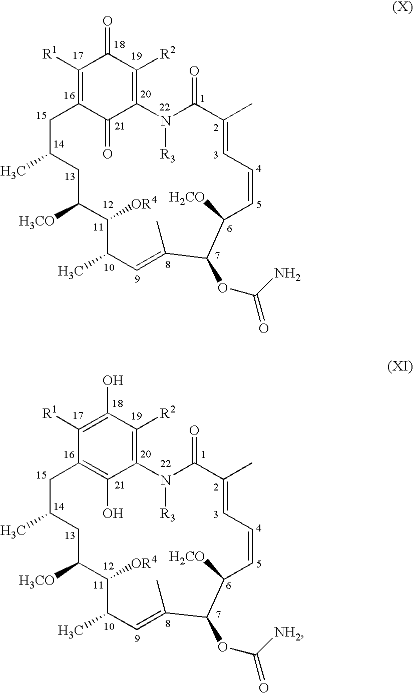

wherein

R¹ is lower alkyl, lower alkenyl, lower alkynyl, optionally substituted lower alkyl, alkenyl, or alkynyl; lower alkoxy, alkenoxy and alkynoxy; straight or branched alkylamines, alkenyl amines and alkynyl amines; a 3-6 member heterocyclic group that is optionally substituted (and R¹ is preferably a 3-6 member heterocyclic ring wherein N is the heteroatom);

R² is H, lower alkyl, lower alkenyl, lower alkynyl, optionally substituted lower alkyl, alkenyl, or alkynyl; lower alkoxy, alkenoxy and alkynoxy; straight and branched alkylamines, alkenyl amines and alkynyl amines; a 3-6 member heterocyclic group that is optionally substituted;

R³ is H, lower alkyl, lower alkenyl, lower alkynyl, optionally substituted lower alkyl, alkenyl, or alkynyl; lower alkoxy, alkenoxy and alkynoxy; straight or branched alkylamines, alkenyl amines, alkynyl amines; or wherein the N is a member of a heterocycloalkyl, heterocylokenyl or heteroaryl ring that is optionally substituted;

R⁴ is H, lower alkyl, lower alkenyl, lower alkynyl, optionally substituted lower alkyl, alkenyl, or alkynyl, and

wherein the ring double bonds between positions C2=C3, C4=C5, and C8=C9 are optionally hydrogenated to single bonds.

In one embodiment the substituents of formulae X and XI are defined as in Xie et al., WO05/095347. The compounds of formulae X and XI may be synthesized as described in Xie et al., WO05/095347.

In a further embodiment the Hsp90 inhibitor is selected from a group comprising the exemplified compounds of formulae X and XI as detailed in Xie et al., WO05/095347.

In one embodiment the Hsp90 inhibitor is a compound of formula XII:

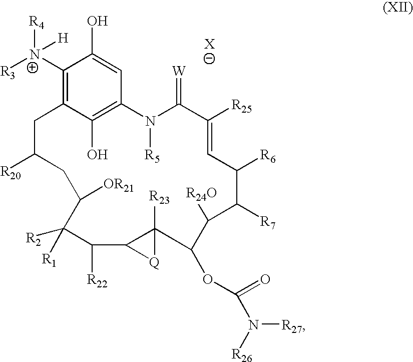

wherein independently for each occurrence:

W is oxygen or sulfur;

Q is oxygen, NR, N(acyl) or a bond;

X⁻ is a conjugate base of a pharmaceutically acceptable acid;

R for each occurrence is independently selected from the group consisting of hydrogen, alkyl, cycloalkyl, heterocycloalkyl, aralkyl, heteroaryl, and heteroaralkyl;

R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;

R₃ and R₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₃ taken together with R₄ represent a 4-8 membered optionally substituted heterocyclic ring;

R₅ is selected from the group consisting of H, alkyl, aralkyl, and a group having the formula

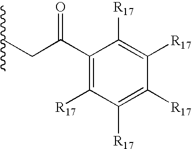

wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₈, —N(R₁₈)CO₂R₁₉, —OC(O)N(R₁₈)(R₁₉), —N(R18)SO₂R₁₉, —N(R₁₈)C(O)N(R₁₈)(R₁₉), and —CH₂O-heterocyclyl;

R₆ and R₇ are both hydrogen; or R₆ and R₇ taken together form a bond;

R₈ is hydrogen, alkyl, alkenyl, alkynyl, aryl, cycloalkyl, heterocycloalkyl, aralkyl, heteroaryl, heteroaralkyl, or —[(CR₂)ₚ]—R₁₆;

R₉ is alkyl, alkenyl, alkynyl, aryl, cycloalkyl, heterocycloalkyl, aralkyl, heteroaryl, heteroaralkyl, or —[(CR₂)ₚ]—R₁₆;

R₁₀ and R₁₁ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₀ and R₁₁ taken together with the nitrogen to which they are bonded represent a 4-8 membered optionally substituted heterocyclic ring;

R₁₂ is alkyl, alkenyl, alkynyl, aryl, cycloalkyl, heterocycloalkyl, aralkyl, heteroaryl, heteroaralkyl, or —[(CR₂)ₚ]—R₁₆;

R₁₃ and R₁₄ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)ₚ]—R₁₆; or R₁₃ and R₁₄ taken together with the nitrogen to which they are bonded represent a 4-8 membered optionally substituted heterocyclic ring;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂ (R¹⁹), —CON(R₁₈)(R₁₉), —OC(O)N(R₁₈)(R₁₉), —SO₂N(R¹⁸)(R¹⁹), —N(R₁₈)(R₁₉), —OC(O)OR₁₈, —COOR₁₈, —C(O)N(OH)(R₁₈), —OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, cycloalkyl, heterocycloalkyl, aralkyl, heteroaryl, and heteroaralkyl;

R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;

R₂₀, R₂₁, R₂₂, R₂₄, and R₂₅, for each occurrence are independently alkyl;

R₂₃ is alkyl, —CH₂OH, —CHO, —COOR₈, or —CH(OR₁₈)₂;

R₂₆ and R₂₇ for each occurrence are independently selected from the group consisting of hydrogen, alkyl, aryl, cycloalkyl, heterocycloalkyl, aralkyl, heteroaryl, and heteroaralkyl;

provided that when R₁ is hydroxyl, R₂ is hydrogen, R₆ and R₇ taken together form a double bond, R₂₀ is methyl, R₂₁ is methyl, R₂₂ is methyl, R₂₃ is methyl, R₂₄ is methyl, R₂₅ is methyl, R₂₆ is hydrogen, R₂₇ is hydrogen, Q is a bond, and W is oxygen; R₃ and R₄ are not both hydrogen nor when taken together represent an unsubstituted azetidine; and

the absolute stereochemistry at a stereogenic center of formula XII may be R or S or a mixture thereof and the stereochemistry of a double bond may be E or Z or a mixture thereof.

In one embodiment the substituents of formula XII are defined as in Adams et al., US2006/0019941. The compounds of formula XII may be synthesized as described in Adams et al., US2006/0019941.

In a further embodiment the Hsp90 inhibitor is selected from a group comprising the exemplified compounds of formula XII as detailed in Adams et al., US2006/0019941.

In one embodiment the Hsp90 inhibitor is a compound of formula XIII:

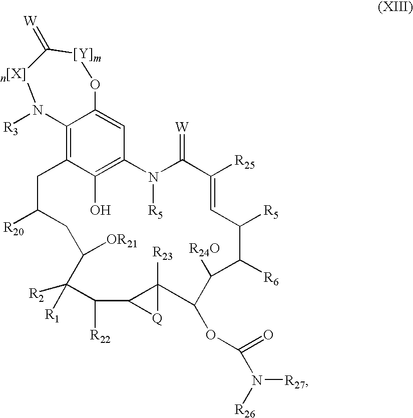

wherein, independently for each occurrence,

W is oxygen or sulfur;

Z is oxygen or sulfur;

Q is oxygen, NR, N(acyl) or a bond;

n is equal to 0, 1, or 2;

m is equal to 0, 1, or 2;

X and Y are independently C(R³⁰)²; wherein R³⁰ for each occurrence is independently selected from the group consisting of hydrogen, alkyl, cycloalkyl, heterocycloalkyl, aralkyl, heteroaryl, and heteroaralkyl; or —[(CR²)^{p}]—R¹⁶;

R for each occurrence is independently selected from the group consisting of hydrogen, alkyl, cycloalkyl, heterocycloalkyl, aralkyl, heteroaryl, and heteroaralkyl;

R¹ is hydroxyl, alkoxyl, —OC(O)R⁸, —OC(O)OR⁹, —OC(O)NR¹⁰R¹¹, —OSO₂R¹², —OC(O)NHSO₂NR¹³R¹⁴, NR¹³R¹⁴, or halide; and R² is hydrogen, alkyl, or aralkyl; or R¹ and R² taken together, along with the carbon to which they are bonded, represent —(C═O)—, —(C═N—OR)—, —(C═N—NHR)—, or —(C═N—R)—;

R³ are each independently selected from the group consisting of hydrogen, alkyl, alkenyl, alkynyl, aryl, cycloalkyl, heterocycloalkyl, aralkyl, heteroaryl, heteroaralkyl, and —[(CR₂)^{p}]—R¹⁶;

R⁴ is selected from the group consisting of H, alkyl, aralkyl, and a group having the formula

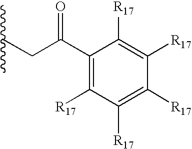

wherein R¹⁷ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR¹⁸, —CO₂R¹⁸, —N(R¹⁸)CO₂R¹⁹, —OC(O)N(R¹⁸)(R¹⁹), —N(R¹⁸)SO₂R¹⁹, —N(R¹⁸)C(O)N(R¹⁸)(R¹⁹), and —CH₂O-heterocyclyl;

R⁵ and R⁶ are both hydrogen; or R⁵ and R⁶ taken together form a bond;

R⁸ is hydrogen, alkyl, alkenyl, alkynyl, aryl, cycloalkyl, heterocycloalkyl, aralkyl, heteroaryl, heteroaralkyl, or —[(CR²)^{p}]—R¹⁶;

R⁹ is alkyl, alkenyl, alkynyl, aryl, cycloalkyl, heterocycloalkyl, aralkyl, heteroaryl, heteroaralkyl, or —[(CR²)^{p}]—R⁶;

R¹⁰ and R¹¹ are each independently selected from the group consisting of hydrogen, alkyl, alkenyl, alkynyl, aryl, cycloalkyl, heterocycloalkyl, aralkyl, heteroaryl, heteroaralkyl, and —[(CR²)^{p}]—R¹⁶; or R¹⁰ and R11 taken together with the nitrogen to which they are bonded represent a 4-8 membered optionally substituted heterocyclic ring;

R¹² is alkyl, alkenyl, alkynyl, aryl, cycloalkyl, heterocycloalkyl, aralkyl, heteroaryl, heteroaralkyl, or —[(CR²)^{p}]—R¹⁶;

R¹³ and R¹⁴ are each independently selected from the group consisting of hydrogen, alkyl, alkenyl, alkynyl, aryl, cycloalkyl, heterocycloalkyl, aralkyl, heteroaryl, heteroaralkyl, and —[(CR²)^{p}]—R¹⁶; or R¹³ and R¹⁴ taken together with the nitrogen to which they are bonded represent a 4-8 membered optionally substituted heterocyclic ring;

R¹⁶ for each occurrence is independently selected from the group consisting of hydrogen, hydroxyl, acylamino, —N(R¹⁸)COR¹⁹, —N(R¹⁸)C(O)OR¹⁹, —N(R¹⁸)SO₂ (R¹⁹), —CON(R¹⁸)(R¹⁹), —OC(O)N(R¹⁸)(R¹⁹), —SO₂N(R¹⁸)(R¹⁹), —N(R¹⁸)(R¹⁹), —OC(O)OR¹⁸, —COOR¹⁸, —C(O)N(OH)(R¹⁸), —OS(O)₂ OR¹⁸, —S(O)₂OR¹⁸, —OP(O)(OR¹⁸)(OR¹⁹), —N(R¹⁸)P(O)(OR¹⁸)(OR¹⁹), and —P(O)(OR¹⁸)(OR¹⁹);

p is 1, 2, 3, 4, 5, or 6;

R¹⁸ for each occurrence is independently selected from the group consisting of hydrogen, alkyl, aryl, cycloalkyl, heterocycloalkyl, aralkyl, heteroaryl, and heteroaralkyl;

R¹⁹ for each occurrence is independently selected from the group consisting of hydrogen, alkyl, aryl, cycloalkyl, heterocycloalkyl, aralkyl, heteroaryl, and heteroaralkyl; or

R¹⁸ taken together with R¹⁹ represent a 4-8 membered optionally substituted ring;

R²⁰, R²¹, R²², R²⁴, and R²⁵, for each occurrence are independently alkyl;

R²³ is alkyl, —CH₂OH, —CHO, —COOR¹⁸, or —CH(OR¹⁸)₂;

R²⁶ and R²⁷ for each occurrence are independently selected from the group consisting of hydrogen, alkyl, aryl, cycloalkyl, heterocycloalkyl, aralkyl, heteroaryl, and heteroaralkyl; and

the absolute stereochemistry at a stereogenic center of formula XIII may be R or S or a mixture thereof and the stereochemistry of a double bond may be E or Z or a mixture thereof.

In one embodiment the substituents of formula XIII are defined as in Adams et al., US2006/0019941. The compounds of formula XIII may be synthesized as described in Adams et al., US2006/0019941.

In a further embodiment the Hsp90 inhibitor is selected from a group comprising the exemplified compounds of formula XIII as detailed in Adams et al., US2006/0019941.

In one embodiment the Hsp90 inhibitor is a compound of formula XIV:

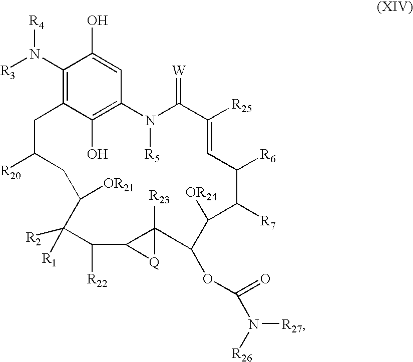

wherein independently for each occurrence:

W is oxygen or sulfur;

Q is oxygen, NR, N(acyl) or a bond;

X— is a conjugate base of a pharmaceutically acceptable acid;

R for each occurrence is independently selected from the group consisting of hydrogen, alkyl, cycloalkyl, heterocycloalkyl, aralkyl, heteroaryl, and heteroaralkyl;

R¹ is hydroxyl, alkoxyl, —OC(O)R⁸, —OC(O)OR⁹, —OC(O)NR¹⁰R¹¹, —OSO₂R¹², —OC(O)NHSO₂ NR¹³R¹⁴, —NR¹³R¹⁴, or halide;

R² is hydrogen, alkyl, or aralkyl; or R¹ and R² taken together, along with the carbon to which they are bonded, are —(C═O)—, —(C═N—OR)—, —(C═N—NHR)—, or —(C═N—R)—;

R³ and R⁴ are each independently selected from the group consisting of hydrogen, alkyl, alkenyl, alkynyl, aryl, cycloalkyl, heterocycloalkyl, aralkyl, heteroaryl, heteroaralkyl, and —[(CR²)^{p}]—R¹⁶; or R³ taken together with R⁴ represent a 4-8 membered optionally substituted heterocyclic ring;

R⁵ is selected from the group consisting of H, alkyl, aralkyl, and a group having the formula

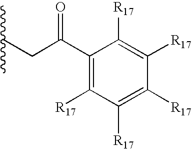

wherein R¹⁷ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR¹⁸, —CO₂R¹⁸, —N(R¹⁸)CO₂R¹⁹, —OC(O)N(R¹⁸)(R1⁹), —N(R¹⁸)SO₂R¹⁹, —N(R¹⁸)C(O)N(R¹⁸)(R¹⁹), and —CH₂O-heterocyclyl;

R⁶ and R⁷ are both hydrogen; or R⁶ and R⁷ taken together form a bond;

R⁸ is hydrogen, alkyl, alkenyl, alkynyl, aryl, cycloalkyl, heterocycloalkyl, aralkyl, heteroaryl, heteroaralkyl, or —[(CR²)^{p}]—R¹⁶;

R⁹ is alkyl, alkenyl, alkynyl, aryl, cycloalkyl, heterocycloalkyl, aralkyl, heteroaryl, heteroaralkyl, or —[(CR²)^{p}]—R¹⁶;

R¹⁰ and R¹¹ are each independently selected from the group consisting of hydrogen, alkyl, alkenyl, alkynyl, aryl, cycloalkyl, heterocycloalkyl, aralkyl, heteroaryl, heteroaralkyl, and —[(CR²)^{p}]—R¹⁶; or R¹⁰ and R¹¹ taken together with the nitrogen to which they are bonded represent a 4-8 membered optionally substituted heterocyclic ring;

R¹² is alkyl, alkenyl, alkynyl, aryl, cycloalkyl, heterocycloalkyl, aralkyl, heteroaryl, heteroaralkyl, or —[(CR²)^{p}]—R¹⁶;

R¹³ and R¹⁴ are each independently selected from the group consisting of hydrogen, alkyl, alkenyl, alkynyl, aryl, cycloalkyl, heterocycloalkyl, aralkyl, heteroaryl, heteroaralkyl, and —[(CR²)^{p}]—R¹⁶; or R¹³ and R¹⁴ taken together with the nitrogen to which they are bonded represent a 4-8 membered optionally substituted heterocyclic ring;

R¹⁶ for each occurrence is independently selected from the group consisting of hydrogen, hydroxyl, acylamino, —N(R¹⁸)COR¹⁹, —N(R¹⁸)C(O)OR¹⁹, —N(R¹⁸)SO₂ (R¹⁹), —CON(R¹⁸)(R¹⁹), —OC(O)N(R¹⁸)(R¹⁹), —SO₂N(R¹⁸)(R¹⁹), —N(R¹⁸)(R¹⁹), —OC(O)OR¹⁸, —COOR¹⁸, —C(O)N(OH)(R¹⁸), —OS(O)₂ OR¹⁸, —S(O)₂ OR¹⁸, —OP(O)(OR¹⁸)(OR¹⁹), —N(R¹⁸)P(O)(OR¹⁸)(OR¹⁹), and —P(O)(OR¹⁸)(OR¹⁹);

p is 1, 2, 3, 4, 5, or 6;

R¹⁸ for each occurrence is independently selected from the group consisting of hydrogen, alkyl, aryl, cycloalkyl, heterocycloalkyl, aralkyl, heteroaryl, and heteroaralkyl;

R¹⁹ for each occurrence is independently selected from the group consisting of hydrogen, alkyl, aryl, cycloalkyl, heterocycloalkyl, aralkyl, heteroaryl, and heteroaralkyl; or R¹⁸ taken together with R¹⁹ represent a 4-8 membered optionally substituted ring;

R²⁰, R²¹R²², R²⁴, and R²⁵, for each occurrence are independently alkyl;

R²³ is alkyl, —CH₂OH, —CHO, —COOR¹⁸, or —CH(OR¹⁸)₂;

R²⁶ and R²⁷ for each occurrence are independently selected from the group consisting of hydrogen, alkyl, aryl, cycloalkyl, heterocycloalkyl, aralkyl, heteroaryl, and heteroaralkyl;

provided that when R¹ is hydroxyl, R² is hydrogen, R⁶ and R⁷ taken together form a double bond, R²⁰ is methyl, R²¹ is methyl, R²² is methyl, R²³ is methyl, R²⁴ is methyl, R²⁵ is methyl, R²⁶ is hydrogen, R²⁷ is hydrogen, Q is a bond, and W is oxygen; R³ and R⁴ are not both hydrogen nor when taken together represent an unsubstituted azetidine; and

the absolute stereochemistry at a stereogenic center of formula XIV may be R or S or a mixture thereof and the stereochemistry of a double bond may be E or Z or a mixture thereof.

In one embodiment the substituents of formula XVI are defined as in Tong et al., WO07/002,093. The compounds of formula XVI may be synthesized as described in Tong et al., WO07/002,093.

In a further embodiment the Hsp90 inhibitor is selected from a group comprising the exemplified compounds of formula XVI as detailed in Tong et al., WO07/002,093.

In one embodiment the Hsp90 inhibitor is a compound of formula XV:

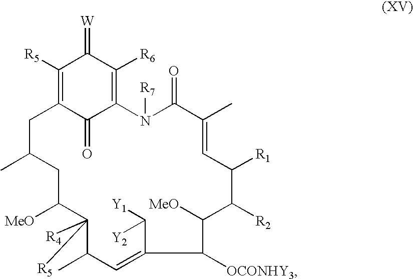

wherein

W represents: O or N—OH, N—O—COR₁₀, or N—O—X—R₁₁; wherein

X represents substituted or unsubstituted (C₁-C₁₀) alkyl or (C₁-C₁₀) alkenyl or (C₁₆-C₁₀) aryl; R₁₁ represents hydrogen, hydroxyl, halogen, cyanide, or CON(R₈)(R₉), N(R₈)(R₉), CO₂R₁₀ wherein R₈ and R₉ are independently selected from the group consisting of H, optionally substituted amine, optionally substituted (C₁-C₂₀) alkyl, optionally substituted (C₂-C₂₀ heteroalkyl, optionally substituted (C₂-C₂₀) alkenyl, optionally substituted (C₂-C₂₀) heteroalkenyl, optionally substituted (C₂-C₂₀) alkenyl, optionally substituted aryl, optionally substituted heteroaryl, optionally substituted arylalkyl, optionally substituted heteroarylalkyl, optionally substituted cycloalkyl, and optionally substituted cycloheteroalkyl; or R₈ is selected from the group consisting of H, optionally substituted C₁-C₆ alkyl, optionally substituted C₅-C₈ aryl, and optionally substituted C₅-C₈ heteroaryl; or R₈, together with R₉ forms an optionally substituted 4-7 membered heterocyclic or carbocyclic ring;

R₁₀ is selected from the group of H, optionally substituted (C₁-C₂₀) alkyl, optionally substituted (C₁-C₂₀) heteroalkyl, optionally substituted (C₂-C₂₀) alkenyl, optionally substituted (C₂-C₂₀) heteroalkenyl, optionally substituted (C₁-C₂₀) alkynyl, optionally substituted (C₆-C₂₀) aryl, optionally substituted (C₆-C₂₀) aryl, optionally substituted (C₃-C₂₀) heteroaryl, optionally substituted (C₇-C₂₀) arylalkyl, optionally substituted (C₄-C₂₀) heteroarylalkyl, optionally substituted (C₃-C₂₀) cycloalkyl, optionally substituted (C₂-C₂₀) cycloheteroalkyl;

R₁ and R₂ are both hydrogen or R₁ and R₂ together form a single bond; R₃, R₄), R₆, R-₇, Y₁, Y₂, Y₃ are independently selected from the group consisting of H, halo, —OH, O-alkyl, O-acetyl, —O-aryl, OC(O)R₁₀, —SO₂—R₁₀, OR¹⁰ and —NHR¹⁰, or together form oxo (═O), or hydroxylamino alkoxyimine or aryloxyimine, thioketo; or R₃ and R⁴ or Y₁ and Y₂ form a heterocyclic residue selected from the group consisting of aziridinyl, azetidinyl, pyrrolidinyl, piperidinyl, thiazolidinyl, oxazolidinyl, morpholino, piperazinyl, 4-(C₁-C₄) alkylpiperidinyl and N—(C₁-C₄) piperazinyl; where said alkyl, phenyl and naphthyl groups may be substituted with one or more residues selected from the group consisting of (C₁-C₈) alkyl, halo, nitro, amino, azido and (C₁-C₈) alkoxyl;

R₅ is independently selected from the group consisting of optionally substituted (C₁-C₂₀) alkyl, optionally substituted (C₁-C₂₀) heteroalkyl, optionally substituted (C₂-C₂₀) alkenyl, optionally substituted (C₂-C₂₀) heteroalkenyl, optionally substituted (C₂-C₂₀) alkynyl, optionally substituted (C₆-C₂₀) aryl, optionally substituted (C₃-C₂₀) heteroaryl, optionally substituted (C₇-C₂₀) arylalkyl, optionally substituted (C₄-C₂₀) heteroarylalkyl, optionally substituted (C₃-C₂₀) cycloalkyl, optionally substituted (C₂-C₂₀) cycloheteroalkyl, N(R₈)(R₉), —OR₁₀, —SR₁₀, —N(R₉)—C(O)R₁₀, —N(R₈)—C(O)—OR₁₀, —N(R₈)—C(O)—N(R₈)(R₁₀), —N(R₈)—C(S)OR₁₀, —N(R₈)—C(S)—OR₁₀, —N(R₈)—C(S)—NR₈R₁₀, wherein R₈, R₉, and R₁₀ are defined as above; and

When W is O, R₅ is NH—OH, NH—O—COR₁, or NH—O—X—R₁₁, wherein X, R¹⁰ and R₁₁ are defined as the above

It is intended that where both R₁₁ and R₅ incorporate one or more of R₈, R₉ or R₁₀, that each occurrence of R₈, R₉, or R₁₀ may be independently selected and may be the same or different from other occurrences of R₈, R₉ and R₁₀.

In one embodiment the substituents of formula XV are defined as in Tao et al., WO07/059,116. The compounds of formula XV may be synthesized as described in Tao et al., WO07/059,116.

In a further embodiment the Hsp90 inhibitor is selected from a group comprising the exemplified compounds of formula XV as detailed in Tong et al., WO07/002,093.

In one embodiment the Hsp90 inhibitor is a compound of formulae XVI, XVII and XVIII:

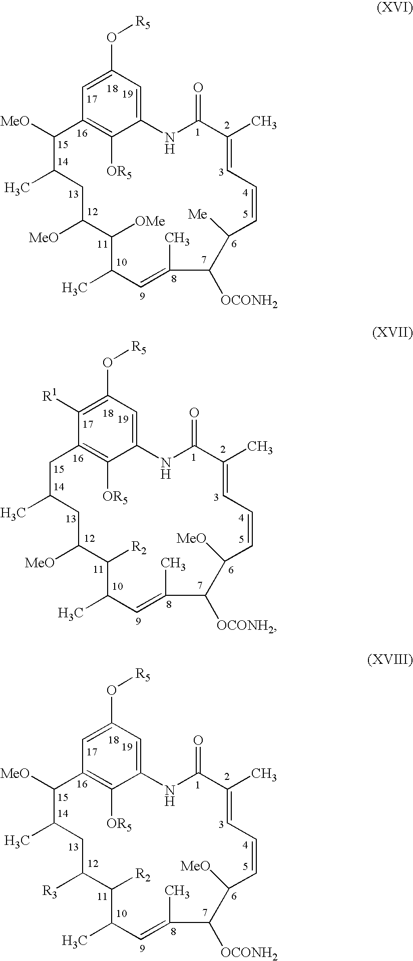

R₁ represents H, OH, OMe, —NHCH₂ CH═CH₂ or —NHCH₂ CH₂N(CH₃)₂;

R₂ represents OH, or keto;

R₃ represents OH or OMe;

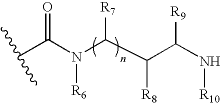

R₅ represents H or
wherein:

n represents 0 or 1; R₆ represents H, Me, Et or iso-propyl;

R₇, R₈ and R₉ each independently represent H or a C1-C4 branched or linear chain alkyl group; or R₇ and R₈, or R₈ and R₉, may be connected so as to form a 6-membered carbocyclic ring;

R₁₀ represents H or a C1-C4 branched or linear chain alkyl group; provided however that the R₅ moieties are not both H and that when neither R₅ moiety represents H then the two R₅ moieties are the same.

The above structure shows a representative tautomer and the invention embraces all tautomers of the compounds of formula XVI, XVII, and XVIII for example keto compounds where enol compounds are illustrated and vice versa.

In one embodiment the substituents of formulae XVI, XVII and XVIII are defined as in Guiblin et al., WO07/026,027. The compounds of formulae XVI, XVII and XVIII may be synthesized as described in Guiblin et al., WO07/026,027.

In a further embodiment the Hsp90 inhibitor is selected from a group comprising the exemplified compounds of formulae XVI, XVII and XVIII as detailed in Guiblin et al., WO07/026,027.

In one embodiment the Hsp90 inhibitor is a compound of formula XIX:

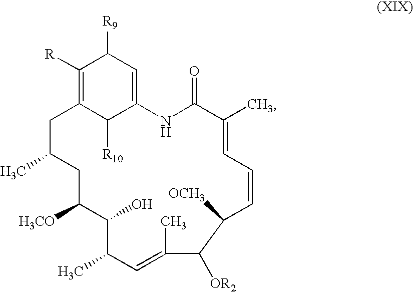

wherein R is methoxy or an R⁶R⁵N amine, where R⁵ and R⁶ are independently H, C₁-C₈ alkyl, C₁-C₈ hydroxyalkyl, C₂-C₈ alkenyl, C₂-C₈ alkynyl, cycloalkyl, heterocyclo, aryl, or heteroaryl; or R⁵ and R⁶ and the nitrogen to which they are attached combine to form a substituted or unsubstituted 3, 4, 5, 6, or 7 membered ring; wherein R² is an aminoacyl group with 1 to 6 carbon atoms, an acyl group with a phenyl moiety (i.e., a benzoyl group), or an acyl group with an alkyl or cycloalkyl moiety comprising 3 to 4 carbon atoms; and wherein R⁹ and R¹⁰ are either hydroxy groups or keto groups and amine or amino salts thereof.

In one embodiment the substituents of formula XIX are defined as in Wenkert et al., WO07/098,229. The compounds of formula XIX may be synthesized as described in Wenkert et al., WO07/098,229.

In a further embodiment the Hsp90 inhibitor is selected from a group comprising the exemplified compounds of formula XIX as detailed in Wenkert et al., WO07/098,229.

In one embodiment the Hsp90 inhibitor is a compound of formula XX:

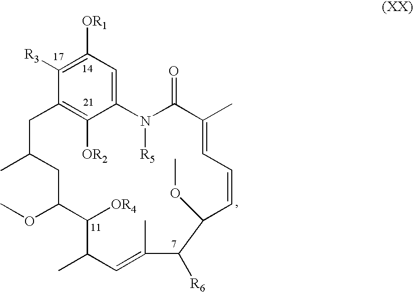

wherein

R₁ and R₂ are independently H, C₁₋₆ alkyl, C₃₋₈cycloalkyl, C(═O)C₁₋₁₀ alkyl, C(═O)(CH₂)ₙ-cycloalkyl, C(═O)(CH₂)ₙ-aryl, wherein n=1-10, alkoxy, alkylthiol, glycoside, glucuronide or sulfate, C(═O)CH(X)NH₂, and C(═O)CH(X)OH, wherein X=an amino acid side chain;

R₃ is H, NHCH₂CH═CH₂ NHCH₂CH₂N(CH₃)₂, NHCH₂CH₂NC₄H₈, azetidinyl, furfuryl, morpholinyl, piperazinyl, piperidinyl, piperazinyl, pyrrolidinyl, tetrahydrofurfuryl, 2-methyl-1-aziridinyl, (dimethylamino)methyl-1-aziridinyl, 3-(dimethylamino)-1-azetidinyl, 3-hydroxy-1-pyrrolidinyl, 3,4-dihydroxy-1-pyrrolidinyl, or NR₇, R₈, OR₇, SR₇, wherein R₇ and R₈ are independently H, C₁₋₁₀ alkyl, alkenyl, alkynyl, alkoxy, alkylhalide, alkyldihalide, amine, cycloalkyl, carboxyalkyl, (acetylamino)alkyl, (dimethylamino)alkyl, 1-(methoxymethyl)alkyl, 2-(1,3-dioxolan-2-yl)alkyl, 4,4-dimethoxybutyl, [[(1,1-dimethylethoxy)carbonyl]amino]alkyl, [[(1,1-dimethylethoxy)carbonyl]alkylamino]alkyl, 1-(hydroxymethyl)alkyl, 1-(hydroxymethyl)-2-methylalkyl, 2-(hydroxymethyl)cycloalkyl, (diethylamino)alkyl, 2-(dimethylamino)-1-methylethyl, (ethylmethylamino)alkyl, [(2-fluoroethyl)methylamino]alkyl, [(2,2-difluoroethyl)methylamino]alkyl, [bis(2-hydroxyethyl)amino]alkyl, (dimethyloxidoamino)alkyl, (trimethylammonio)alkyl, (1-aziridinyl)alkyl, (1-aziridinylmethyl)alkyl, (1-azetidinyl)alkyl, (2-deoxy-D-glucos-2-yl), (6-deoxy-D-glucos-6-yl), (1H-imidazol-4-yl)alkyl, (1-methyl-1H-imidazol-4-yl)alkyl, (1-methyl-1H-imidazol-5-yl)alkyl, (4-morpholinyl)alkyl, (4-pyridinyl)alkyl, (1-piperidinyl)alkyl, (1-piperazinyl)alkyl, (1-pyrrolidinyl)alkyl, (1-ethyl-2-pyrrolidinyl)methyl, or 2-(N-methyl-pyrrolidin-2-yl)ethyl; and wherein when R₁ and R₂ are both H, R₃ is not OCH₃ or NH₂;

R₄ and R₅ are independently H, C₁₋₆ alkyl, C₃₋₈ cycloalkyl, C(═O)C₁₋₁₀ alkyl, C(═O)(CH₂)ₙ-aryl, C(═O)(CH₂)ₙ-cycloalkyl, alkoxy, alkylthiol, glycoside, glucuronide or sulfate, wherein n=1-10; and,

R₆ is O, OC(═O)NH₂C(═O)C₁₀ alkyl, OSO₂OH, OC(═O)OSO₂OH and OC(═O)NR₉R₁₀ wherein R₉ and R₁₀ are independently H and C₁₀ alkyl.

In one embodiment the substituents of formula XX are defined as in Ross et al., WO06/098761. The compounds of formula XX may be synthesized as described in Ross et al., WO06/098761.

In a further embodiment the Hsp90 inhibitor is selected from a group comprising the exemplified compounds of formula XX as detailed in Ross et al., WO06/098761.

In one embodiment the Hsp90 inhibitor is a compound of formula XXI:

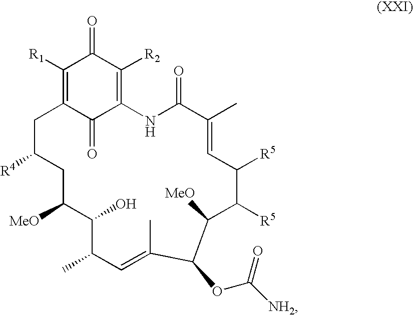

wherein R¹ is MeO, (CH₂)₃ N or R⁹—NH, wherein R⁹ is selected from the group consisting of H, substituted or unsubstituted C₁-C₆ alkyl, substituted or unsubstituted C₁-C₆ alkenyl, substituted or unsubstituted C₁-C₆ alkynyl, substituted or unsubstituted C₃-C₆ cycloalkyl, piperidinyl, N-alkylpiperidinyl, hexahydropyranyl, furfuryl, tetrahydrofurfuryl, pyrrolidinyl, N-alkylpyrrolidinyl, piperazinylamino, N-alkylpiperazinyl, morpholinyl, N-alkylaziridinylmethyl, (1-azabicyclo[1.3.0]hex-1-yl)ethyl, 2-(N-methyl-pyrrolidin-2-yl)ethyl, 2-(4-imidazolyl)ethyl, 2-(1-methyl-4-imidazolyl)ethyl, 2-(1-methyl-5-imidazolyl)ethyl, 2-(4-pyridyl)ethyl, and 3-(4-morpholino)-1-propyl, or R⁶ is H and R¹ and R⁵ taken together form a group of the formula NH-Z-0, wherein Z is a linker comprised of from 1 to 6 carbon atoms and 0 to 2 nitrogen atoms and wherein the 0 is attached at the position of R⁵; R² is selected from the group consisting of H, halogen, OR¹⁰, NHR¹⁰, SR¹⁰, aryl, and heteroaryl, wherein R¹⁰ is selected from the group consisting of substituted or unsubstituted C₁-C₆ alkyl, substituted or unsubstituted C₁-C₆ alkenyl, substituted or unsubstituted C₁-C₆ alkynyl, and substituted or unsubstituted C₃-C₆ cycloalkyl; R³ is H, OH, or OMe; R⁴ is H or Me; R⁵ is OH or O—C(═O)—CH₂NH₂ and R is H, or R⁵ and R⁶ taken together form ═O or ═N—OR¹¹, wherein R¹¹ is selected from the group consisting of H, substituted or unsubstituted C₁-C₆ alkyl, substituted or unsubstituted C₁-C₆ alkenyl, substituted or unsubstituted C₁-C₆ alkynyl, substituted or unsubstituted C₃-C₆ cycloalkyl, aryl, or heteroaryl; R⁷ is H and R⁸ is H or OH, or R⁷ and R⁸ taken together form a bond; and X is O or a bond, with the provisos that when R³ is H, R⁴ is Me, and R⁷ is H and R⁸ is H or R⁷ and R⁸ taken together form a bond that either R⁶ is H and R¹ and R⁵ taken together form a group of the formula NH-Z-O, wherein Z is a linker comprised of from 1 to 6 carbon atoms and 0 to 2 nitrogen atoms and wherein the O is attached at the position of R⁵, or that R¹ is (CH₂)₃ N or R⁹—NH, wherein R⁹ is selected from the group consisting of piperidinyl, N-alkylpiperidinyl, hexahydropyranyl, furfuryl, tetrahydrofurfuryl, pyrrolidinyl, N-alkylpyrrolidinyl, piperazinylamino, N-alkylpiperazinyl, morpholinyl, N-alkylaziridinylmethyl, (1-azabicyclo [1.3.0]hex-1-yl)ethyl, 2-(N-methyl-pyrrolidin-2-yl)ethyl, 2-(4-imidazolyl)ethyl, 2-(1-methyl-4-imidazolyl)ethyl, 2-(1-methyl-5-imidazolyl)ethyl, 2-(4-pyridyl)ethyl, and 3-(4-morpholino)-1-propyl; and that when R³ is H and R⁴ is Me that R⁷ is H and R⁸ is OH.

In one embodiment the substituents of formula XXI are defined as in Santi et al., US2003/0114450. The compounds of formula XXI may be synthesized as described in Santi et al., US2003/0114450.

In a further embodiment the Hsp90 inhibitor is selected from a group comprising the exemplified compounds of formula XXI as detailed in Santi et al., US2003/0114450.

### Macbecins

In one embodiment the Hsp90 inhibitor is Macbecin I, Macbecin II or derivatives thereof. Macbecin I and II were isolated from the culture broth of Nocardia sp. No. C-14919. Macbecins I and II belong to the ansamycin group and have a benzoquinone and hydroquinone nucleus, respectively. (Ono et al., Gann. 1982, 73, 938-944; Muroi et al., J. Antibiot. (Tokyo) 1980, 33, 205-212; Tanida et al., J. Antibiot. (Tokyo) 1980, 33, 199-204).

Herbimycins

In one embodiment the Hsp90 inhibitor is Herbimycin A or a derivative thereof. Herbimycin A is a benzochinoid ansamycin antibiotic isolated from Streptomyces sp. MH237-CF8, which specifically inhibits the phosphorylation of tyrosine residues catalyzed by various protein kinases. Omura et al., J. Antibiot. (Tokyo) 1979, 32, 255-261). Derivatives of herbimycin have also been described, including 8,9-Epoxide, 7,9-cyclic carbamate, 17 or 19-amino derivatives, halogenated and other related derivatives; see, e.g., Shibata et al., J. Antibiot. (Tokyo) 1986, 39, 415-423; Shibata et al., J. Antibiol. (Tokyo) 1986, 39, 1630-1633; and Oikawa et al., Biol. Pharm. Bull. 1994, 17, 1430-1432.

Tyrosine Hydroxylase Modulators

In one embodiment the tyrosine hydroxylase modulator is metirosine or Demser.

In another embodiment the tyrosine hydroxylase modulator is 3,5-diiodo-4-hydrobenzoic acid.

Methods of Screening for Agents Capable of Treating or Preventing a Substance-Related Disorder

Methods for identifying Hsp90 inhibitors and modulators, using target based screening, structure-based rational design, and high throughput screening, are known in the art. See, e.g., Neckers et al., Curr. Med. Chem. 2003, 10, 733-739; Rowlands et al., Anal. Biochem. 2004, 327, 176-183; Dymock et al., Exp. Op. Ther. Patents 14 2004, 837-847; Workman et al., Cancer Letters 2004, 206, 149-157; Aherne et al., Methods Mol. Med. 2003, 85, 149-161; Chiosis et al., Curr. Cancer Drug Targets 2003, 3, 371-376; Khan et al., Biochem. J. 2008, 409(2):581-9; or Carreras et al., Anal. Biochem. 2003, 317, 40-46 and references cited therein.

The present invention also provides methods of identifying an agent capable of treating or preventing a substance-related disorder. In certain embodiments, the methods comprise the step of identifying an agent capable of modulating interaction between Hsp90 and tyrosine hydroxylase.

The agent can be identified by any technique apparent to those of skill in the art to be useful for identifying an agent capable of reducing interaction between two polypeptides. In certain embodiments, agent can be identified by contacting tyrosine hydroxylase, in a composition comprising Hsp90, and measuring tyrosine hydroxylase activity. For instance, compositions comprising tyrosine hydroxylase and Hsp90 can be contacted with a candidate agent and a corresponding change in tyrosine hydroxylase activity can be assessed. In certain embodiments, the change can be compared to the corresponding change in a composition that lacks substantial amounts of Hsp90. Changes in tyrosine hydroxylase activity, can be measured by any technique apparent to one of skill in the art. Exemplary techniques are provided in the examples below.

In one embodiment, the method of identifying an agent comprises determining a first level of tyrosine hydroxylase activity in a cell or tissue that expresses tyrosine hydroxylase, contacting said cell or tissue with a test agent, then determining a second level of tyrosine hydroxylase in said cell or tissue. A difference in the first level and second level of tyrosine hydroxylase activity is indicative of the ability of the test agent to modulate tyrosine hydroxylase activity. In one embodiment, an agent may have agonistic activity if the second level of tyrosine hydroxylase activity is greater than the first level of tyrosine hydroxylase activity. In certain embodiments, agonistic activity comprises at least about a 2, 4, 6, 8, 10, or greater fold increase in the second level of tyrosine hydroxylase activity compared to the first level of tyrosine hydroxylase activity. In another embodiment, an agent may have antagonistic activity if the second level of tyrosine hydroxylase activity is less than the first level of tyrosine hydroxylase activity. In certain embodiments, antagonistic activity comprises at least about a 2, 4, 6, 8, 10, or greater fold decrease in the second level of tyrosine hydroxylase activity compared to the first level of tyrosine hydroxylase activity.

In one embodiment, the method of identifying an agent comprises determining a first level of tyrosine hydroxylase activity in a cell or tissue that expresses tyrosine hydroxylase and Hsp90, contacting said cell or tissue with a test agent, then determining a second level of tyrosine hydroxylase in a corresponding cell or tissue that does not express Hsp90. A difference in the first level and second level of tyrosine hydroxylase activity is indicative of the ability of the test agent to modulate tyrosine hydroxylase and Hsp90 interaction. In one embodiment, an agent may have agonistic activity if the second level of tyrosine hydroxylase activity is less than the first level of tyrosine hydroxylase activity. In certain embodiments, agonistic activity comprises at least about a 2, 4, 6, 8, 10, or greater fold decrease in the second level of tyrosine hydroxylase activity compared to the first level of tyrosine hydroxylase activity. In another embodiment, an agent may have antagonistic activity if the second level of tyrosine hydroxylase activity is greater than the first level of tyrosine hydroxylase activity. In certain embodiments, antagonistic activity comprises at least about a 2, 4, 6, 8, 10, or greater fold increase in the second level of tyrosine hydroxylase activity compared to the first level of tyrosine hydroxylase activity.

The present invention also provides methods of identifying agents that specifically bind to a tyrosine hydroxylase-Hsp90 complex. The invention thus provides assays to detect compounds that specifically bind to a hydroxylase-Hsp90 complex. Test agents can be contacted with a hydroxylase-Hsp90 complex under conditions conducive to binding, and compounds that specifically bind to a hydroxylase-Hsp90 complex are identified. Methods that can be used to carry out the foregoing are commonly known in the art.

In some embodiments, cell free assays utilizing a purified hydroxylase-Hsp90 complex may be performed to identify agents. Putative modulators of a hydroxylase-Hsp90 complex may be identified by assaying hydroxylase activity in the presence of varying concentrations of the agent and examining the extent of phosphate incorporation into a suitable substrate.

By way of example, diversity libraries, such as random or combinatorial peptide or nonpeptide libraries can be screened for agents. Many libraries are known in the art that can be used, e.g., chemically synthesized libraries, recombinant (e.g., phage display libraries), and in vitro translation-based libraries.

Examples of chemically synthesized libraries are described in Fodor et al., Science 251:767-773 (1991); Houghten et al., Nature 354:84-86 (1991); Lam et al., Nature 354:82-84 (1991); Medynski, Bio/Technology 12:709-710 (1994); Gallop et al., J Medicinal Chemistry 37(9):1233-1251 (1994); Ohlmeyer et al., Proc. Natl. Acad. Sci. U.S.A. 90:10922-10926 (1993); Erb et al., Proc. Natl. Acad. Sci. U.S.A. 91:11422-11426 (1994); Houghten et al., Biotechniques 13:412 (1992); Jayawickreme et al., Proc. Natl. Acad. Sci. U.S.A. 91:1614-1618 (1994); Salmon et al., Proc. Natl. Acad. Sci. U.S.A. 90:11708-11712 (1993); PCT Publication No. WO 93/20242; and Brenner and Lerner, Proc. Natl. Acad. Sci. U.S.A. 89:5381-5383 (1992).

Examples of phage display libraries are described in Scott and Smith, Science 249:386-390 (1990); Devlin et al., Science, 249:404-406 (1990); Christian, R. B., et al., J. Mol. Biol. 227:711-718 (1992)); Lenstra, J. Immunol. Meth. 152:149-157 (1992); Kay et al., Gene 128:59-65 (1993); and PCT Publication No. WO 94/18318, published Aug. 18, 1994. In vitro translation-based libraries include but are not limited to those described in PCT Publication No. WO 91/05058, published Apr. 18, 1991; and Mattheakis et al., Proc. Natl. Acad. Sci. U.S.A. 91:9022-9026 (1994).

By way of examples of non-peptide libraries, a benzodiazepine library (see e.g., Bunin et al., Proc. Natl. Acad. Sci. U.S.A. 91:4708-4712 (1994)) can be adapted for use. Peptoid libraries (Simon et al., Proc. Natl. Acad. Sci. U.S.A. 89:9367-9371 (1992)) can also be used. Another example of a library that can be used, in which the amide functionalities in peptides have been permethylated to generate a chemically transformed combinatorial library, is described by Ostresh et al., Proc. Natl. Acad. Sci. U.S.A. 91:11138-11142 (1994).

Screening the libraries can be accomplished by any of a variety of commonly known methods. See, e.g., the following references, which disclose screening of peptide libraries: Parmley and Smith, Adv. Exp. Med. Biol. 251:215-218 (1989); Scott and Smith, Science 249:386-390 (1990); Fowlkes et al., Bio/Techniques 13:422-427 (1992); Oldenburg et al., Proc. Natl. Acad. Sci. U.S.A. 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. U.S.A. 89:6988-6992 (1992); Ellington et al., Nature 355:850-852 (1992); U.S. Pat. No. 5,096,815, U.S. Pat. No. 5,223,409, and U.S. Pat. No. 5,198,346; Rebar and Pabo, Science 263:671-673 (1993); and PCT Publication No. WO 94/18318, published Aug. 8, 1994.

In a specific embodiment, screening can be carried out by contacting the library members with tyrosine hydroxylase-Hsp90 complex immobilized on a solid phase and harvesting those library members that bind to the complex. Either or both members of the complex can be immobilized in particular embodiments. Examples of such screening methods, termed “panning” techniques are described by way of example in Parmley and Smith, Gene 73:305-318 (1988); Fowlkes et al., Bio/Techniques 13:422-427 (1992); PCT Publication No. WO 94/18318; and in references cited herein above.

In another embodiment, the two-hybrid system for selecting interacting proteins in yeast (Fields and Song, Nature 340:245-246 (1989); Chien et al., Proc. Natl. Acad. Sci. U.S.A. 88:9578-9582 (1991)) can be used to identify molecules that specifically modulate the interaction of tyrosine hydroxylase and Hsp90.

### 7.3 Methods of Use

In one embodiment provided herein are methods for the treatment or prevention of a substance-related disorder in a subject in need thereof comprising administering to the subject an amount of an Hsp90 inhibitor or modulator or a pharmaceutically acceptable salt thereof, effective to treat or prevent the substance-related disorder.

In one embodiment provided herein are methods for the treatment or prevention of a substance-related disorder in a subject in need thereof comprising administering to the subject a pharmaceutical compostion comprising a pharmaceutically acceptable carrier and a therapeutically effective amount of an Hsp90 inhibitor or modulator and a therapeutically effective amount of a tyrosine hydroxylase modulator, effective to treat or prevent the substance-related disorder.

In preferred embodiments herein the subject is a human.

In certain embodiments the substance causing a substance-related disorder in a subject includes, but is not limited to alcohol, amphetamine or similarly acting sympathomimetics, caffeine, cannabis, cocaine, hallucinogens, inhalants, nicotine, opioids, phencyclidine (PCP) or similarly acting arylcyclohexylamines, sedatives, hypnotics, anxiolytics or medications such as anesthetics, analgesics, anticholinergic agents, anticonvulsants, antihistamines, antihypertensive and cardiovascular medications, antimicrobial medications, anti-parkinsonian medications, chemotherapeutic agents, corticosteroids, gastrointestinal medications, muscle relaxants, nonsteroidal anti-inflammatory medications, other over-the-counter medications, antidepressant medications, and disulfiram. In another embodiment the substance causing the substance-related disorder in a subject includes but is not limited to heavy metals (e.g., lead or aluminium), rat poisons containing strychnine, pesticides containing nicotine, or acetylcholine-esterase inhibitors, nerve gases, ethylene glycol (antifreeze), carbon monoxide, and carbon dioxide. In yet another embodiment the substance causing the substance-related disorder includes, but is not limited to volatile substances or “inhalants”, such as fuel or paint, if they are used for the purpose of becoming intoxicated.

In certain embodiments the substance causing a substance-related disorder in a subject is alcohol.

In certain embodiments the substance-related disorder is alcohol abuse or alcohol withdrawal.

In one embodiment provided herein are methods of ameliorating or eliminating an effect of a substance-related disorder in a subject in need thereof, comprising administering to the subject an amount of an Hsp90 inhibitor or modulator a pharmaceutically acceptable salt thereof, effective to ameliorate or eliminate the effect of the substance-related disorder.

In certain embodiments the effects of a substance-related disorder include, but are not limited to significant impairment or distress caused by a maladaptive pattern of substance use. The significant impairment or distress is manifested including, but not limited to recurrent substance use resulting in a failure to fulfill major role obligations at work, school, or home (e.g. repeated absences or poor work performance related to substance use; substance-related absences, suspensions, or expulsions from school; neglect of children or household); recurrent substance use in situations in which it is physically hazardous (e.g. driving an automobile or operating a machine when impaired by substance use); recurrent substance-related legal problems (e.g., arrests for substance-related disorderly conduct); continued substance use despite having persistent or recurrent social or interpersonal problems caused or exacerbated by the effects of the substance (e.g. arguments with spouse about consequences of intoxication, physical fights).

In an additional embodiment the effects of a substance-related disorder include, but are not limited to those biochemical or behavioral changes that occur as a result of and within a reasonable time frame following the administration of the substance. Different effects can be expected depending on the substance and the dose administered thereof. For example, the effects of low doses of ethanol include locomotor activation whereas the effects of high doses of ethanol include symptoms of alcohol intoxication (for definition of alcohol intoxication, see American Psychiatric Association, Diagnostic Criteria for DSM-IV, Washington D.C 1 2000, p. 214f).

A method for diminishing, inhibiting or eliminating an addiction-related behavior in a subject suffering from a substance-related disorder, comprising administering to the subject an amount of an Hsp90 inhibitor or modulator a pharmaceutically acceptable salt thereof, effective to diminish, inhibit or eliminate the addiction-related behavior.

A method for alleviating or eliminating withdrawal symptoms in a subject suffering from a substance-related disorder comprising administering to the subject an amount of an Hsp90 inhibitor or modulator or a pharmaceutically acceptable salt thereof, effective to alleviate or eliminate the withdrawal symptoms.

In another embodiment representative withdrawal symptoms include, but are not limited to autonomic hyperactivity (e.g. sweating or pulse rate greater than 100); increased hand tremor; insomnia or hypersomnia; nausea or vomiting; transient visual, tactile, or auditory hallucinations or illusions; psychomotor agitation or retardation; anxiety; grand mal seizures; fatigue; vivid, unpleasant dreams; increased appetite or weight gain; dysphoric or depressed mood; irritability, frustration or anger; difficulty concentrating; restlessness; decreased heart rate; sweating; or muscle pain.

A Hsp90 inhibitor or modulator, a pharmaceutical acceptable salt, solvate, hydrate or prodrug thereof, can be administered in any form deemed suitable by a practitioner of skill in the art and by any technique deemed suitable by the same. Exemplary forms and techniques for administration are provided herein.

In one embodiment the Hsp90 inhibitor or modulator is administered to a subject suffering from a substance-related disorder in a dosage range of 0.1 to 1000 mg per day.

In one embodiment the Hsp90 inhibitor or modulator is administered to a subject suffering from a substance-related disorder in a dosage range of 0.1 to 300 mg per day.

In one embodiment the Hsp90 inhibitor or modulator is administered to a subject suffering from a substance-related disorder in a dosage range of 0.1 to 150 mg per day.

In one embodiment the Hsp90 inhibitor or modulator is administered to a subject suffering from a substance-related disorder in a dosage range of 0.1 to 50 mg per day.

In one embodiment the Hsp90 inhibitor or modulator is administered to a subject suffering from a substance-related disorder in a dosage range of 0.1 to 20 mg per day.

The present disclosure provides methods of treating or preventing a substance-related disorder using an Hsp90 inhibitor or modulator or a pharmaceutically acceptable salt, solvate, hydrate or prodrug thereof.

In certain embodiments the Hsp90 inhibitor or modulator may be provided as a pharmaceutically acceptable salt deemed suitable by one of skill in the art. (See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

### 7.4 Pharmaceutical Compositions

A Hsp90 inhibitor or modulator, a pharmaceutically acceptable salt, solvate, hydrate or prodrug thereof, can be administered in any form deemed useful by the practitioners of skill in the art. In certain embodiments, the Hsp90 inhibitor or modulator is administered in a pharmaceutical composition comprising one or more pharmaceutically acceptable carriers, excipients or diluents.

The compound provided herein may be administered alone, or in combination with one or more other compounds provided herein, one or more other active ingredients. The pharmaceutical compositions that comprise a compound provided herein may be formulated in various dosage forms for oral, parenteral, and topical administration. The pharmaceutical compositions may also be formulated as modified release dosage forms, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2003; Vol. 126).

In one embodiment provided herein is a pharmaceutical composition comprising a pharmaceutically acceptable carrier and a therapeutically effective amount of an Hsp90 inhibitor and a therapeutically effective amount of a tyrosine hydroxylase modulator.

In one embodiment provided herein is a pharmaceutical composition comprising a pharmaceutically acceptable carrier and a therapeutically effective amount of an Hsp90 modulator and a therapeutically effective amount of a tyrosine hydroxylase modulator.

In one embodiment, the pharmaceutical compositions are provided in a dosage form for oral administration, which comprise a compound provided herein or a pharmaceutically acceptable salt, solvate, or prodrug thereof; and one or more pharmaceutically acceptable excipients or carriers.

In another embodiment, the pharmaceutical compositions are provided in a dosage form for parenteral administration, which comprise a compound provided herein or a pharmaceutically acceptable salt, solvate, or prodrug thereof; and one or more pharmaceutically acceptable excipients or carriers.

In yet another embodiment, the pharmaceutical compositions are provided in a dosage form for topical administration, which comprise a compound provided herein or a pharmaceutically acceptable salt, solvate, or prodrug thereof; and one or more pharmaceutically acceptable excipients or carriers.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

Certain ansamycin derivatives such as geldanamycin and 17-AAG, can display less than ideal water solubility. 17-DMAG, having an alkyl amino group, can be more soluble.

Useful formulations comprising geldanamycin, 17-AAG or 17-DMAG for pharmaceutical use include:

(a) Tabibi et al., U.S. Pat. No. 6,682,758 disclosed a formulation for a water insoluble drug such as 17-AAG comprising (a) the drug, (b) a water-miscible organic solvent for the drug, (c) a surfactant, and (d) water. The water miscible solvent can be dimethylsulfoxide (DMSO), diπαcethylformamide, ethanol, glycerin, propylene glycol, or polyethylene glycol. The surfactant preferably is a phospholipid (especially egg phospholipid).

(b) Ulm et al., WO 03/086381, which discloses a method for preparing pharmaceutical formulations for ansamycins by (a) providing the ansamycin dissolved in ethanol; (b) mixing the product of step (a) with a medium chain triglyceride to form a first mixture; (c) substantially removing the ethanol from the first mixture; (d) combining the product of step (c) with an emulsifying agent and a stabilizer to form a second mixture; and (e) emulsifying the second mixture. The emulsified second mixture optionally can be lyophilized and then re-hydrated. In a specific combination, the medium chain triglyceride comprises caprylic and/or caproic acid, the emulsifying agent comprises phosphatidylcholine, and stabilizer comprises sucrose.

(c) Ulm et al., WO 04/082676 discloses a pharmaceutical composition comprising an Hsp90 inhibitor such as 17-AAG, an emulsifying agent, and an oil comprising both medium and long chain triglycerides.

(d) Zhong et al., US 2005/0256097 discloses a formulation of 17-AAG in a vehicle comprising (i) a first component that is ethanol; (ii) a second component that is a polyethoxylated castor oil (e.g., Cremophor™); and (iii) optionally a third component that is selected from the group consisting of propylene glycol, PEG 300, PEG 400, glycerol, and combinations thereof.

(e) Tao et al., Am. Assoc. Cancer Res., 96th Annual Meeting (Apr. 16-20, 2005), abstract no. 1435, discloses a nanoparticle albumin bound 17-AAG formulation asserted to be suitable for intravenous administration.

(f) Isaacs et al., WO 06/094029, discloses a pharmaceutical formulation comprising 17-AAG dissolved in a vehicle comprising an aprotic, polar solvent and an aqueous mixture of long chain triglycerides.

(g) Mansfield et al., US 2006/0067953, discloses a pharmaceutical formulation for oral administration, comprising an ansamycin and one or more pharmaceutically acceptable solubilizers, with the proviso that when the solubilizer is a phospholipid, it is present in a concentration of at least 5% w/w of the formulation. Other solubilizers disclosed include polyethylene glycols of various molecular weights, ethanol, sodium lauryl sulfate, Tween 80, Solutol® HS 15, propylene carbonate, and so forth. Both dispersion and solution embodiments are disclosed.

(h) Desai et al., WO 06/034147, discloses the use of dimethylsorbide as a solvent for formulating poorly water-soluble drugs such as ansamycins.

(i) Licari et al., 11/595,005, discloses nanoparticulate formulations of 17-AAG and a preferred polymorph of 17-AAG for use in such formulations.

(j) Yu et al., WO 07/084,233 discloses the use of d-alpha-tocopheryl succinate and polyethylene glycol in conjunction with a pharmaceutically acceptable, water-miscible organic solvent.

A person skilled in the art should be able to formulate other ansamycin analogs of the instant disclosure not specifically mentioned in the citations presented hereinabove by applying nothing but ordinary skill in the art using the information in said publications and the references cited therein.

### 8. EXAMPLES

### Example 1

### Tyrosine Hydroxylase (TH) Immunoreactivity Level Upon Prolonged Exposure of Cells to Ethanol

The following example measured up-regulation of TH in cells upon prolonged exposure to ethanol.

Materials and Methods

Recombinant human GDNF polypeptide and anti-GDNF monoclonal neutralizing antibodies were purchased from R&D Systems. Bisindolylmaleimide I (Bis), PP2 and H89 were purchased from EMD Calbiochem. Phosphatase Inhibitor Cocktails 1 & 2, ibogaine-HCl, phosphatidylinositol phospholipase C (PI-PLC), cycloheximide (CHX), Rp-cyclic 3′,5′-hydrogen phosphorothioate adenosine triethylammonium salt (Rp-cAMPS) and anti-TH antibody were purchased from Sigma. Anti-heat shock protein 90αβ (HSP90) and anti-Actin antibodies were purchased from Santa Cruz Biotechnology. Anti-Akt1/2 antibody was purchased from Cell Signaling Technology. Protein G agarose was purchased from Invitrogen. Geldanamycin (GA) was purchased from Alexis Biochemicals. Redivue™ Pro-Mix™ L-[35S] in vitro Cell Labeling Mix was purchased from GE Healthcare. The protease inhibitor cocktail was purchased from Roche Applied Science. Reverse Transcription System and 2. PCR Master Mix were purchased from Promega Corporation. Primers for PCR were synthesized by Sigma-Genosys.

Cell culture. SH-SY5Y human neuroblastoma cells were cultured in the growth medium Dulbecco's modified Eagle medium (DMEM) containing 10% fetal bovine serum (FBS) plus 1. MEM nonessential amino acid solution (Invitrogen). All experiments were carried out in cells that had been incubated in a low serum medium containing 1% FBS for 2 days. GDNF stable cells overexpressing GDNF were derived from SH-SY5Y cells stably transfected with pUSEGDNF and control cells were obtained by stable transfection with the pUSE empty vector (30). The stable cell lines were maintained in growth media containing 500 μg/ml G418 and incubated in low serum media for 2 days prior to experiments.

Treatments. Cells were treated with ethanol for 12-48 h in low serum media by replacing the media with ethanol every 12 h. GDNF or ibogaine was added to the media for the last 12 h of 24-h treatments with ethanol, and the inhibitors GA, Bis, PP2, H89 and RpcAMPS were added for the last 9 h of the 24-h ethanol treatment. PI-PLC was incubated with cells at a concentration of 0.3 u/ml for 1 h as described previously (1). Anti-GDNF neutralizing antibodies were dissolved in PBS and were used at a concentration of 10 μg/ml.

Reverse transcription-polymerase chain reaction (RT-PCR). Total RNA isolation was carried out using TRIzol reagent according to the manufacturer's protocol. RNA samples were analyzed by RT-PCR with Actin as an internal control as described previously (1,30). After the RT reaction, PCR was run for 32 cycles with the TH primers as follows: upstream 5′-TTC GCG CAG TTC TCG CAG GAC ATT GGC-3′ and downstream 5′-CGT GTA CGG GTC GAA CTT CAC GGA GAA-3′.

Western blot and immunoprecipitation. Cells were collected and lysed in RIPA buffer plus protease and phosphatase inhibitor cocktails for Western blot and immunoprecipitation as described previously (1,30). Briefly, 15 μg of each homogenate was subjected to an SDS-PAGE gel and blotted on a nitrocellulose membrane for Western blot analysis with anti-TH antibody or other primary antibodies, followed by reprobing with anti-Actin antibody. Alternatively, 500 μg of each homogenate was incubated with 5 μg of anti-TH, anti-HSP90 or anti-Akt antibodies in TBS-T buffer overnight at 4° C.1 followed by a 2-h incubation with Protein G agarose beads. Immunoprecipitates were separated on an SDS-PAGE gel for Western blot analysis.

Pulse-chase analysis. 24 h after ethanol treatment, cells were labeled following a pulse-chase procedure as described previously (31) with modifications. Briefly, cells were incubated with methionine/cystine-free DMEM (Invitrogen) containing 1% dialyzed FBS for 1 h and then labeled in the Met/Cysfree medium containing 25 μCi of Amersham Redivue Pro-Mix L-[35S] in vitro Cell Labeling Mix per ml for 3 h. Dialysis of FBS was performed against the Met/Cys-free medium overnight. After labeling, cells were washed twice with the normal medium and chased in the normal medium without ethanol for 2-8 h before homogenizing as described above. Homogenates were centrifuged and protein concentrations of the supernatants were determined. An equal amount of protein from each sample was immunoprecipitated with anti-TH antibody and separated by electrophoresis on an 8% SDS gel. Autoradiography was performed by exposure of the dried gels to Amersham Storage Phosphor Screen and visualization of radioactive signals using the Typhoon 9410 Variable Mode Imager (GE Healthcare).

Quantification and statistical analysis. Signals from Western blot, RT-PCR or ³⁵S labeled radioactivity were quantified using NIH Image 1.61 as described previously (He et al., J. Neurosci. 2005, 25, 619-628 and He et al., Faseb J 2006, 20, 2420-2422). Density of TH immunoreactivity was used to estimate TH protein levels that were expressed as ratios to those of Actin. TH mRNA levels were expressed as ratios to those of Actin. In the pulse-chase analysis, percentage of the radioactive signals of ³⁵S labeled TH during the chase times was used to estimate relative stability of the protein. Results were obtained from at least three separate experiments and the statistical significance of differences between treatments and control was analyzed using a one-sample t-test (http://www.graphpad.com/quickcalcs/OneSampleT1.cfm).

Experiment. Tyrosine hydroxylase (TH) catalyzes the hydroxylation of L-tyrosine to L-3,4-dihydroxyphenylalanine, which is the ratelimiting step in the biosynthesis of catecholamine neurotransmitters, including dopamine (Nagatsu et al., J. Biol. Chem. 1964, 239, 2910-2917; Levitt et al., J. Pharmacol. Exp. Ther. 1965, 148, 1-8). The mesolimbic dopamine system, which consists of the dopaminergic neurons in the ventral tegmental area (VTA) and projections to the nucleus accumbens and the prefrontal cortex, is the major neural structure that mediates the rewarding effects of drugs of abuse and ethanol. Biochemical adaptations in dopaminergic midbrain neurons induced by chronic exposure to drugs of abuse have been observed and implicated in relation to drug addiction (Self et al., Annu. Rev. Neurosci. 1995, 18, 463-495; Nestler et al., J. Neurosci. 1992, 12, 2439-2450; Koob et al., Trends Pharmacol. Sci. 1992, 13, 177-184; Wise et al., Annu. Rev. Psychol. 1989, 40, 191-225).

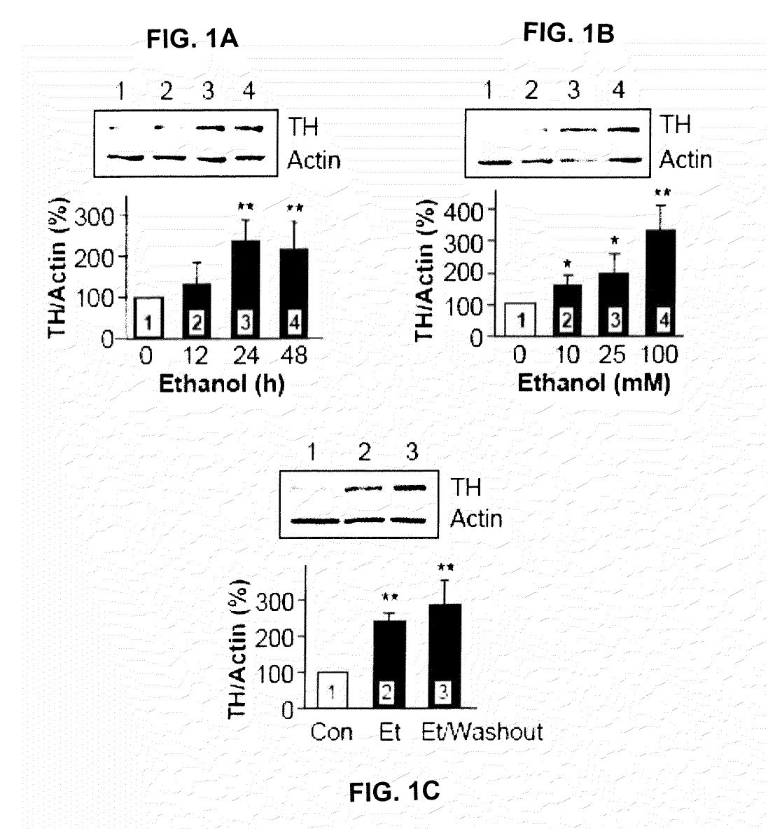

It was first tested whether the chronic ethanol-mediated increase in TH immunoreactivity could be mimicked in the dopaminergic-like SH-SY5Y cells. It was found that prolonged ethanol treatment resulted in an increase in TH protein levels after 24 h and 48 h of exposure to ethanol (FIG. 1A). The increase in TH protein levels by ethanol occurred in a dose-dependent manner (FIG. 1B). Interestingly, even 10 mM, a low, nonintoxicating dose of ethanol, produced a significant increase in the immunoreactivity of TH. Finally, the ethanol-induced increase in TH protein levels was long-lasting and could be detected 24 h after ethanol washout (FIG. 1C).

These results indicated that the increase in TH levels is a long-lasting adaptation to ethanol exposure, which can be detected in SH-SY5Y cells.

### Example 2

### Prolonged Exposure of Cells to Ethanol Enhances the Stability of TH Protein

The present example measured whether prolonged ethanol exposure induced an increase in transcription of the TH gene or synthesis of the protein.

For materials and methods please refer to Example 1.

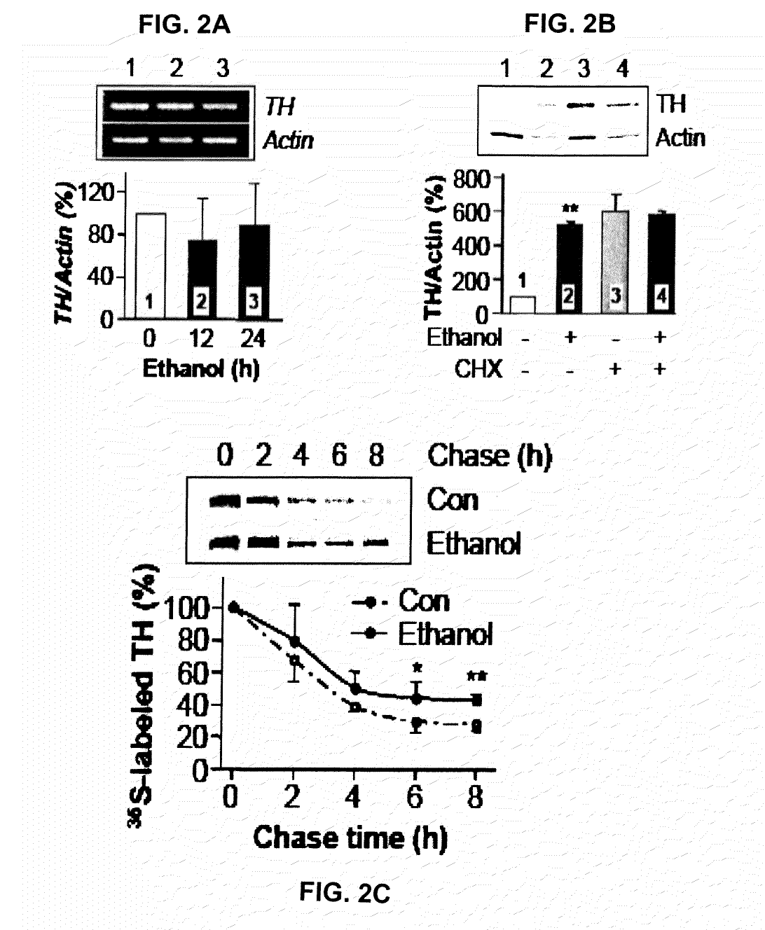

It was found that prolonged incubation with ethanol did not increase TH mRNA (FIG. 2A). In addition, cycloheximide (CHX), a protein synthesis inhibitor, did not inhibit ethanol-mediated increases in TH protein levels (FIG. 2B). These results indicate that ethanol increases TH protein levels in a transcription- and translation-independent manner, suggesting that ethanol exposure may enhance the stability of the TH protein, resulting in its accumulation. To confirm our conclusion that prolonged exposure of cells to ethanol leads to the stabilization of the TH protein, we performed a pulse-chase analysis. After 24 h ethanol treatment, cells were labeled with ³⁵S-methionine/cysteine and chased in normal medium without ethanol.

Results from autoradiography of ³⁵S-labeled TH protein 5 revealed an increase in the quantity of labeled TH protein after ethanol treatment at all points in the chase time course, as compared to control (FIG. 2C), suggesting that ethanol treatment enhanced the stability of the protein.

### Example 3

### Prolonged Ethanol Exposure Enhances TH Protein Stability Via Heat Shock Protein 90 (Hsp90)

The present example measured the influence of Hsp90 on the increase of ethanol mediated TH immunoreactivity.

For materials and methods please refer to Example 1.

To elucidate the mechanism by which ethanol increases TH protein stability, it was tested whether Hsp90 contributes to the ethanol-mediated increase in TH immunoreactivity. Hsp90 is a molecular chaperone that has extensively been shown to promote the stability and function of many signaling proteins (Zhang et al., J. Mol. Med. 2004, 82, 488-499). Specifically, Hsp90 enhances the stability of proteins by forming an ATP-dependent complex with proteins such as steroid receptors, epidermal growth factor receptor (EGF-R), Her-2, Akt, Raf-1 kinase, p53 and cdk4, protecting them from proteasome-dependent degradation (Zhang et al, supra; Pearl, et al., Annu. Rev. Biochem. 2006, 75, 271-294). Disruption of the complex of Hsp90 with these proteins by Hsp90 inhibitors, such as geldanamycin (GA), leads to protein degradation (Zhang et al, supra; Xiao et al., Curr. Med. Chem. 2007, 14(2), 223-232; Hadden et al., Curr. Top. Med. Chem. 2006, 6(11), 1173-1182; Ochel et al., Cell Stress Chaperones 2001, 6(2), 105-11). GA specifically inhibits the Hsp90 molecular chaperone (Whitesell et al., Mol. Endocrinol. 1996, 10, 705-712; Whitesell et al., Proc. Natl. Acad. Sci. USA, 1994, 91, 8324-8328) by binding to the ATPbinding site in the chaperone (Prodromou et al., Cell 1997, 90, 65-75; Grenert et al., J. Biol. Chem. 1997, 272, 23843-23850). It was hypothesized that if Hsp90 activity contributes to ethanol-induced increases in TH protein levels, GA should block ethanol's action.

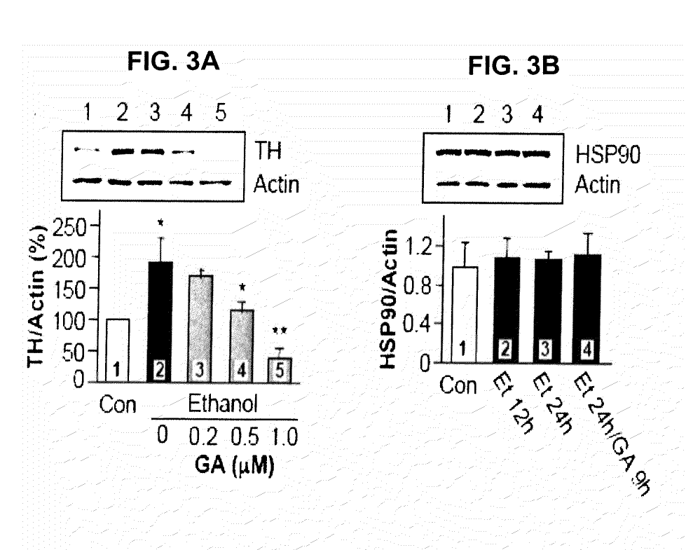

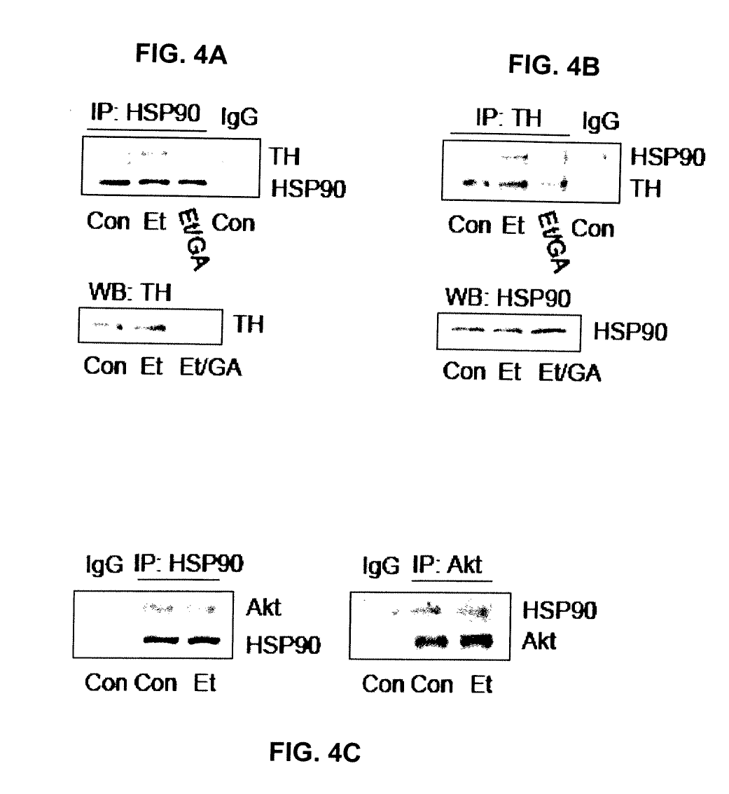

As shown in FIG. 3A, application of GA dose-dependently inhibited the ethanolmediated increase in TH immunoreactivity. It was further found that treatment with ethanol alone, or together with GA, did not alter the protein level of Hsp90 (FIG. 3B). This suggests that the increase in TH protein levels by ethanol treatment is not due to an alteration of Hsp90 protein levels, and that GA treatment specifically inhibits ethanol's induction of TH protein levels. To further test whether Hsp90 participates in maintaining TH stability upon ethanol treatment, the possible association of TH and Hsp90 in untreated and ethanol-treated cells by coimmunoprecipitation with anti-TH or anti-Hsp90 antibodies was measured. As shown in FIGS. 4A and 4B, ethanol treatment for 24 h induced an association of TH with Hsp90, and this association was blocked in the presence of GA. This effect of ethanol is specific for the association between TH and Hsp90 because the ethanol treatment did not alter the association between Akt kinase and Hsp90 (FIG. 4C).

The combined results of Examples 1-3 indicated that prolonged exposure of cells to ethanol specifically induced the formation of a complex of Hsp90 and TH and thus stabilized the TH protein. The examples also demonstrated that the Hsp90 inhibitor geldanamycin inhibited the ethanol-mediated induction of the TH-Hsp90 association.

### Example 4

### GDNF Decreases Ethanol-Mediated Stabilization of TH Protein by Inhibition of the Association of TH and Hsp90

The present example demonstrates that GDNF (glial cell line derived neurotropic factor) decreases ethanol-mediated stabilization of TH protein by inhibition of the association of TH and Hsp90.

For materials and methods please refer to Example 1.

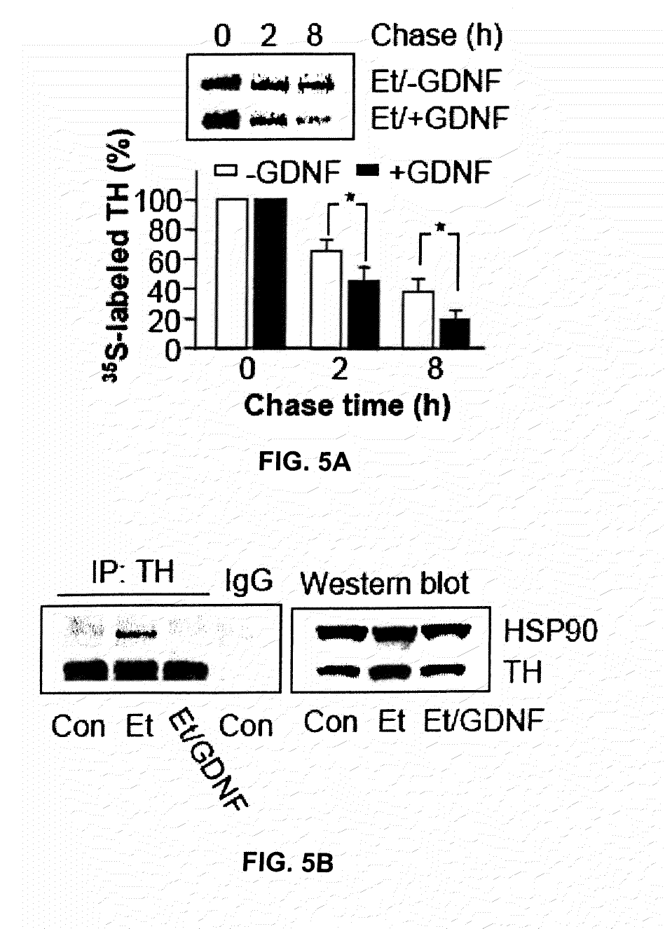

The hypothesis that GDNF reverses the stabilization and association of TH with Hsp90 induced by ethanol treatment was tested. In the presence of ethanol, addition of GDNF downregulates the quantity of ³⁵S-labeled TH at all points on the chase time course (FIG. 5A), indicating that GDNF decreases the stabilization of the TH protein. Furthermore, as shown in FIG. 5B, ethanol exposure for 24 h increased TH immunoreactivity (right panel) and its association with Hsp90 (left panel). Addition of GDNF reversed both the ethanol-mediated increase in TH levels (FIG. 5B, right panel) and the association of Hsp90 with TH (FIG. 5B, left panel), which was not due to alteration in Hsp90 immunoreactivity (FIG. 5B, right panel).

### Example 5

### Ibogaine Reverses Ethanol-Mediated Increases in TH Protein Levels Via GDNF

The present example demonstrates that Ibogaine reverses ethanol-mediated increases in TH protein levels via GDNF.

For materials and methods please refer to Example 1.

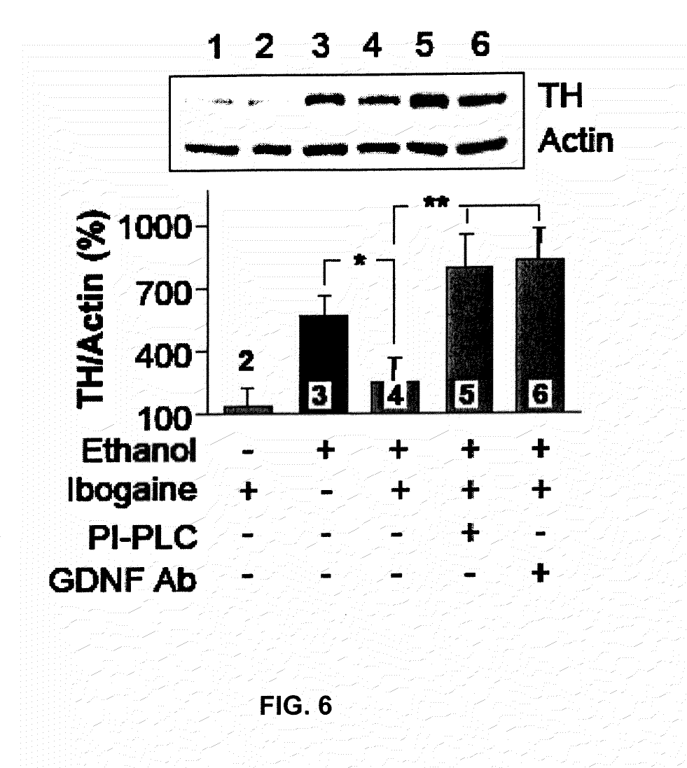

It was previously reported that treatment with ibogaine increases GDNF gene expression and upregulates GDNF-mediated signaling both in vitro in SH-SY5Y cells and in the midbrain region containing the VTA in vivo (He et al., J. Neurosci. 2005, 25, 619-628; He et al., Faseb J. 2006, 20, 2420-2422). To demonstrate that ibogaine, like GDNF, reverses the ethanol-mediatedinduction of TH protein levels SH-SY5Y cells were treated with ethanol for 24 h in the absence and presence of ibogaine, which was added for the last 12 h of the 24-h ethanol treatment. It was found that the addition of ibogaine inhibited ethanol induced increases in TH levels (FIG. 6). In order to confirm that this action of ibogaine is indeed mediated via GDNF, two approaches were applied to block the GDNF signaling pathway and measured ibogaine's effects on TH protein levels. It was found that incubation of cells with ibogaine after treatment with PIPLC (which hydrolyzes the glycosylphosphatidylinositol link of the GDNF coreceptor GFRα1 to the membrane (Jing et al., Cell 1996, 85, 1113-1124), or in the presence of anti-GDNF inhibitory antibodies (which act as GDNF scavengers (Messer et al., Neuron 2000, 26, 247-257; Xu et al., J Neurochem. 1998, 70, 1383-1393), inhibited ibogaine's action on the ethanol-mediated induction of TH immunoreactivity (FIG. 6).

These results indicate that the GDNF signaling pathway mediates ibogaine's ability to negatively regulate this biochemical adaptation to ethanol exposure. These results indicate that GDNF reduces the ethanol-mediated increases in TH protein levels by preventing the association of Hsp90 with TH.

### Example 6

### The Effect of an Hsp90 Inhibitor or Modulator on Ethanol and Sucrose Self-Administration

The present example demonstrates the effect of a Hsp90 inhibitor or modulator on ethanol and sucrose self-administration. Different groups of animals are trained to self administer either 10% ethanol or 5% sucrose. Rats are housed individually and food is always available in the home cage.

Ethanol and Sucrose Self-Administration:

Before the beginning of operant self-administration training, rats are exposed to a 10% ethanol (10E) solution as the only liquid source in their home cages for 3 days. Self-administration training sessions are initiated by the extension of two levers. Each response at the active lever(s) is reinforced by 0.1 ml of solution paired with the illumination of a white cue light above the lever for 5 sec and a 5 sec tone. Responses at the inactive lever are recorded as a measure of nonspecific behavioral activation but have no programmed consequences. Subjects trained to lever-press for oral ethanol for the self-administration and reinstatement procedures undergo a brief period (21 hour) of water restriction during the initial training of the operant response (5 days maximum). Previous studies (M. P. Arolfo, L. Yao, A. S. Gordon et al., Alcohol Clin Exp Res 2004, 28 (9), 1308; H. Nie and P. H. Janak, Psychopharmacology (Berl) 2003, 168 (1-2), 222) have shown that similar water restriction schedules before the beginning of operant self administration does not compromise animals' health. Hughes et al. (J. E. Hughes, H. Amyx, J. L. Howard et al., Laboratory animal science 1994, 44 (2), 135) showed that rats deprived of fluid for 21 hr/day for 3 months did not differ from ad libitum controls with respect to weight loss, organ and tissue appearance at necropsy, hematologic examination or clinical chemical analysis.

Rats quickly learn to consume much, if not all, of their daily fluid needs in a short, restricted period (reviewed by H. L. Evans, Neurotoxicology and Teratology 1990, 12 (5), 531). Rats are monitored daily for weight loss and signs of dehydration. After initial overnight water restriction, rats are placed in operant chambers for a 14 hr overnight session on an FR1 schedule (one reward for every lever press) with 10% sucrose (10S) as the reinforcer and both levers active. Animals are kept on 1 hr water restriction for the next 5 days during which they receive one 45 min session per day on an FR1 schedule with 10S as the reinforcer and one active lever. They are then given free access to water in their home cages for the remainder of the experiment and are trained for 2-3 more sessions with 10S as the reinforcer. The next day, sessions are shortened to 30 minutes and the ratio of responding is increased to FR3. Operant self-administration is initiated according to the sucrose fading technique as described above (Samson, Alcohol Clin. Exp. Res. 1986, 10, 436-42) with minor modifications. Ten percent ethanol (10E) is then be added to the 10% sucrose solution (10S/10E) and the rats receive 3-4 sessions of this solution. Over the next 8-10 sessions, the sucrose concentration is gradually decreased (i.e. 5, 2, and finally 0%) after which the rats have 20-22 sessions with 10E only until a stable baseline consumption is achieved (no more than 20% variation between sessions over 3 consecutive days, in excess of 0.3 g/kg). A separate group of animals is trained to self administer 5% sucrose, this group of animals will serve as a control group. Following approximately two weeks of daily 30 min self-administration sessions with 10% ethanol or 5% sucrose as the reinforcer, animals are injected with either vehicle or a dose of an Hsp90 inhibitor or modulator (1-80 mg/kg i.p.) immediately prior to the self-administration session to determine the drug effects on 10% ethanol or 5% sucrose self-administration. Each group of animal receives only one drug, but receives multiple doses of the same drug over a period of 4 weeks in random order, with one injection per week.

Operant Self Administration of 10% Ethanol.

This protocol is the same as described for 10% ethanol self-administration. Once a stable baseline consumption is achieved (no more than 20% variation between sessions over 3 consecutive days, in excess of 0.3 g/kg), rats are food deprived to begin nicotine self-administration training.

Nose-poke Training. In order to motivate animals to learn a second behavior (nose-poking) they require a short period of food restriction in the home cage so that food delivery can be used as a reward for learning the nose poke behavior. Rats are food restricted (animals have access to 20 g of pellet food per day in the home cage) for 1 week and if any animal falls below 90% of their basal weight food pellets are returned. The general health and well being of each animal is monitored daily and any signs of distress such as changes in behavior, coat condition, eye color and individual body weights are measured. If there are any signs of distress food pellets are returned. During the week of food restriction the animals are trained to nose-poke for food rewards in daily sessions in the operant behavior chambers. Food rewards are predicated on animal responses. A response in the left nose-poke hole results in immediate delivery of a food pellet paired with the illumination of a white, cue light directly above the nose-poke hole for 5 sec. Food pellets (45 mg, Bioserve, San Diego, Calif., USA) are delivered to a food trough situated between the two nose-poke holes. A response on the right nose-poke hole is recorded but elicits no programmed consequence. Levers previously associated with ethanol delivery is not extended during nose-poke, food training sessions. Water is available ad libitum in the home cage.

Drug Tests. The effects of a single injection of one dose of an Hsp90 inhibitor or modulator are tested during self administration of ethanol. An Hsp90 inhibitor or modulator or vehicle is administered 15 min prior to the test sessions. Drug dose test order is based on a within subjects Latin square design.

### Example 7

### The Effect of an Hsp90 Inhibitor or Modulator on Reinstatement of Drug-Seeking

The present example measures the effect of an Hsp90 inhibitor or modulator on reinstatement of drug-seeking.

Following the testing of an Hsp90 inhibitor or modulator on operant self-administration as described above, animals undergo extinction procedures, in which pressing of the active lever produces no reinforcers and no visual/audio stimuli. This signals to the animal that ethanol is no longer available, and the animal stops pressing the active lever when placed into the operant chamber. Then one of the following reinstatement paradigm is used.

Context-induced reinstatement: Subjects are trained to self-administer solutions of either ethanol (4.5% or 10%, v/v), sucrose (5%, g/v), beer (4.5% ethanol v/v) or near beer on a continuous fixed-ratio-1 (FR1) schedule of reinforcement (FR-1), as previously described. The experiment consists of three phases: acquisition/maintenance in either the first or the second context (random and counterbalanced assignment, referred to as context A), extinction in the other context (referred to as context B) and reinstatement in the acquisition/maintenance context (A) and the extinction, context (B). Animals are tested for operant response reinstatement with no reward availability in the acquisition context (context A) the day after the last extinction session, 2 and 3 weeks later. Animals are also tested in the extinction context (context B) 15 days after the last extinction session (C. Burattini, T. M. Gill, G. Aicardi et al., Neuroscience 2006, 139 (3), 877). Food and water are available ad libitum in their home cages. Animals are injected with either vehicle or a dose of an Hsp90 inhibitor or modulator prior to the reinstatement session. As before, each group of animal receives only one drug but receives multiple doses of the same drug over a period of 4 weeks in random order, with one injection per week. All reinstatement sessions are conducted once per week.

Extinction of Ethanol Self Administration. Rats continue with daily self-administration sessions but without reinforcers available. During extinction sessions all visual (light stimuli) and auditory (tone) cues are absent. Extinction sessions continue until animals reach a predetermined set of criteria pertaining to nose poke and lever press responses.

Reinstatement of Ethanol Self Administration. On reinstatement test days animals are placed in the chambers and are presented with contingent cues previously associated with ethanol and nicotine delivery. Animal response activity is recorded but no rewards are delivered. Reinstatement sessions occur once weekly with extinction sessions in between test days.

Drug Tests. The effects of a single injection of one dose of an Hsp90 inhibitor or modulator is tested during two phases of the experiment: (1) extinction and (2) reinstatement an Hsp90 inhibitor or modulator or vehicle is administered 15 min prior to the test sessions. Drug dose test order is based on a within subjects Latin square design.

### Example 8

### The Effect of an Hsp90 Inhibitor or Modulator on Ethanol Sensitivity Including Loss of Righting Reflex and Blood Ethanol Clearance

The present example measures the effect of an Hsp90 inhibitor or modulator on ethanol sensitivity including loss of righting reflex and blood ethanol clearance.

Loss of Righting Reflex (LORR) (C57BL/6J): This procedure provides an objective measure of the sedating effects of ethanol by examining the length of time mice lay “unconscious” (i.e., unable to right themselves after being placed on their backs) after receiving a high dose of ethanol. These experiments determine if the candidate drugs enhance or reduce the sedating effects of high doses of ethanol. Mice are housed with free access to food and water at all times. Animals are administered vehicle or a dose of an Hsp90 inhibitor or modulator (1-80 mg.kg i.p) ten minutes prior to injection of ethanol (3.2 or 3.6 g/kg i.p.). After the ethanol injection, mice are placed on their backs, and the time taken to lose the righting reflex as well as to regain the righting reflex are measured. Because repeated administration of ethanol at high doses can result in acute tolerance to the loss of righting reflex, the analysis of multiple doses of candidate drugs, or multiple doses of ethanol, is not performed within the same animal.

Blood Ethanol Clearance (Long Evans and Wistar rats): Any drug that reduces voluntary ethanol consumption or sedation could perhaps do so by altering the metabolism or clearance of ethanol from the bloodstream. To determine if an Hsp90 inhibitor or modulator alter blood ethanol clearance, animals are housed with free access to food and water and are administered either vehicle or the most effective dose of an Hsp90 inhibitor or modulator in altering ethanol consumption or sedation (derived from experiments above) 10 min prior to the administration of ethanol (3.6 g/kg). Then, at 10, 30, 90, 180 and 270 min following the ethanol injection, a small nick is made in the lateral portion of the base of the tail of each rat, and approximately 20 ul of blood is collected into a heparinized capillary tube and later analyzed for ethanol content. Each animal is then euthanized immediately following the last blood collection time point by CO2 inhalation.

### Example 9

### Determination of Hsp90 Binding Via In Vitro Assays

The present example demonstrates how in vitro Hsp90 inhibitor strength can be measured.

Hsp90 in Malachite Green Assay. Hsp90 protein is obtained from a commercial source, such as Stressgen (Cat#SPP-770). Assay buffer: 100 mM Tris-HCl, Ph7.4, 20 mM KCl, 6 mM MgCl₂. Malachite green (0.0812% w/v) (M9636) and polyviny alcohol USP (2.32% w/v) (P 1097) are obtained from commercial sources, such as Sigma. A Malachite Green Assay (see Aherne et al., Methods Mol. Med. 2003, 85, 149-161 for method details) is used for examination of ATPase activity of Hsp90 protein. Briefly, Hsp90 protein in assay buffer (100 mM Tris-HCl, Ph7.4, 20 mM KCl, 6 mM MgCl 2) is mixed with ATP alone (negative control) or in the presence of Geldanamycin (a positive control) or a compound of the invention, in a 96-well plate. Malachite green reagent is added to the reaction. The mixtures is incubated at 37° C. for 4 hours and sodium citrate buffer (34% w/v sodium citrate) was added to the reaction. The plate is read by an ELISA reader with an absorbance at 620 nm.

### Example 10

### Systemic Injection of the Hsp90 Inhibitor 17-AAG (50 mg/kg) Reduces Ethanol Consumption and Preference

The present example demonstrates the effect of an Hsp90 inhibitor on voluntary alcohol intake and preference.

Upregulation of tyrosine hydroxylase (the rate limiting enzyme in dopamine synthesis protein level) is a hallmark of the biochemical adaptations to in vivo chronic exposure to drugs of abuse and alcohol. Using the dopaminergic-like SH-SY5Y cells (Biedler et al., 1978, Cancer Res. 38:3751-3757) as a model system, alcohol treatment can result in increased association of tyrosine hydroxylase with heat shock protein 90 (Hsp90) leading to enhanced stability of the protein and its subsequent accumulation (See Examples 1-3).

To test whether the inhibition of Hsp90 activity can reverse behavioral adaptations to long-term exposure to alcohol, an Hsp90 inhibitor was systemically administered to rats, and voluntary alcohol intake and preference was measured. A 20% ethanol intermitten-access paradigm in which the rats consume large quantities of alcohol was used (Carnicella et al., 2008, Proc. Natl. Acad. Sci. USA 105:8114-8119; Carnicella et al., 2009, Alcohol 43:35-43. These references are hereby incorporated by reference in their entireties.).

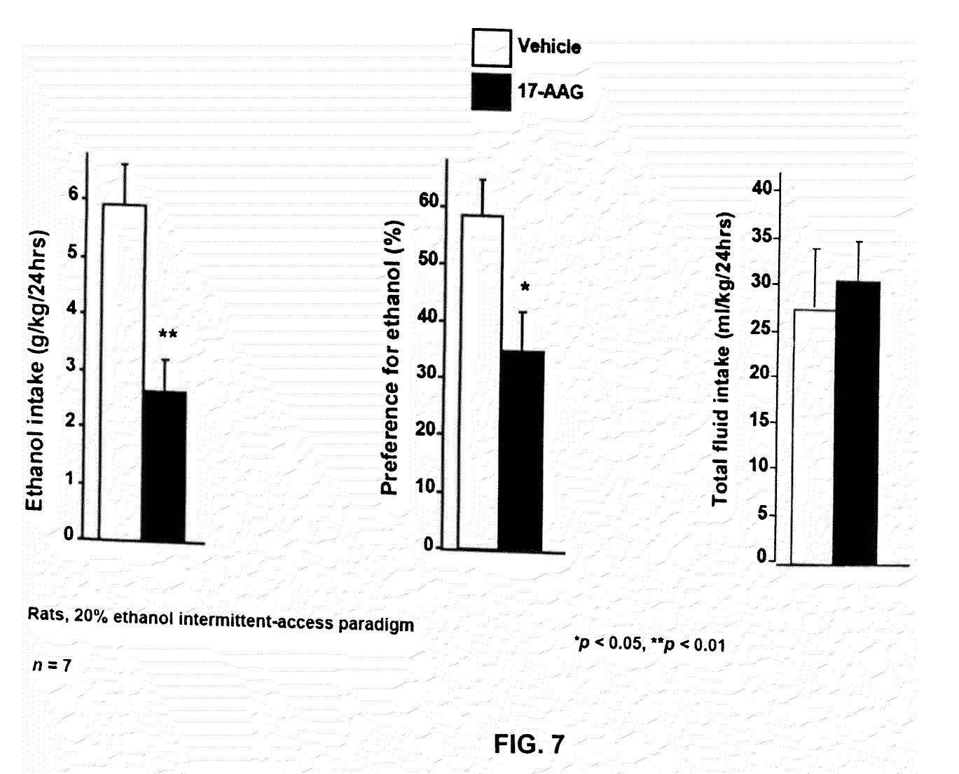

Systemic administration of the Hsp90 inhibitor, 17-AAG (50 mg/kg), significantly reduced ethanol consumption (g/kg/24 hrs) and preference for ethanol (%), as compared to administration of vehicle (FIG. 7). Importantly, water consumption was unaltered, as measured by total fluid intake (ml/kg/24 hrs).

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

What is claimed is:

1. A method for the treatment or prevention of a substance-related disorder in a subject in need thereof comprising administering to the subject an amount of an Hsp90 inhibitor or modulator or a pharmaceutically acceptable salt thereof, effective to treat or prevent the substance-related disorder.

2. A method of ameliorating or eliminating an effect of a substance-related disorder in a subject in need thereof, comprising administering to the subject an amount of an Hsp90 inhibitor or modulator a pharmaceutically acceptable salt thereof, effective to ameliorate or eliminate the effect of the substance-related disorder.

3. A method of claim 2, wherein the effect of a substance-related disorder is significant impairment or distress caused by a maladaptive pattern of substance use.

4. A method for diminishing, inhibiting or eliminating an addiction-related behavior in a subject suffering from a substance-related disorder, comprising administering to the subject an amount of an Hsp90 inhibitor or modulator a pharmaceutically acceptable salt thereof, effective to diminish, inhibit or eliminate the addiction-related behavior.

5. A method for alleviating or eliminating withdrawal symptoms in a subject suffering from a substance-related disorder comprising administering to the subject an amount of an Hsp90 inhibitor or modulator or a pharmaceutically acceptable salt thereof, effective to alleviate or eliminate the withdrawal symptoms.

6. The method of any one of claims 1, 2, 4 or 5 wherein the substance comprises alcohol, amphetamine or similarly acting sympathomimetics, caffeine, cannabis, cocaine, hallucinogens, inhalants, nicotine, opioids, phencyclidine (PCP) or similarly acting arylcyclohexylamines, sedatives, hypnotics, anxiolytics or medications such as anesthetics, analgesics, anticholinergic agents, anticonvulsants, antihistamines, antihypertensive and cardiovascular medications, antimicrobial medications, anti-parkinsonian medications, chemotherapeutic agents, corticosteroids, gastrointestinal medications, muscle relaxants, nonsteroidal anti-inflammatory medications, other over-the-counter medications, antidepressant medications, and disulfiram.

7. The method of any one of claims 1, 2, 4 or 5 wherein the substance is alcohol.

8. The method of any one of claims 1, 2, 4 or 5 wherein the substance-related disorder is an alcohol-related disorder.

9. The method of any one of claims 1, 2, 4 or 5 wherein the Hsp90 inhibitor is a compound of any one of formulae II to XXI.

10. The method of any one of claims 1, 2, 4 or 5 wherein the Hsp90 inhibitor is selected from a group comprising the exemplified compounds of any one of formulae II to XXI.

11. The method of any one of claims 1, 2, 4 or 5 wherein the Hsp90 inhibitor is geldanamycin, 17-allylamino-17-demethoxygeldanamycin (17-AAG) or 17-dimethyaminoethylamino-17-demethoxygeldanamycin (17-DMAG).

12. The method of any one of claims 1, 2, 4 or 5 wherein the Hsp90 inhibitor or modulator is administered at 0.1 to 1000 mg per day.

13. The method of any one of claims 1, 2, 4 or 5 wherein the Hsp90 inhibitor or modulator is administered at 0.1 to 300 mg per day.

14. The method of any one of claims 1, 2, 4 or 5 wherein the Hsp90 inhibitor or modulator is administered at 0.1 to 150 mg per day.

15. The method of any one of claims 1, 2, 4 or 5 wherein the Hsp90 inhibitor or modulator is administered at 0.1 to 50 mg per day.

16. The method of any one of claims 1, 2, 4 or 5 wherein the Hsp90 inhibitor or modulator is administered at 0.1 to 20 mg per day.

17. A method for the treatment or prevention of a substance-related disorder in a subject in need thereof comprising administering to the subject an amount of an Hsp90-TH modulator or a pharmaceutically acceptable salt thereof, effective to treat or prevent the substance-related disorder.

18. The method of claim 17 wherein the Hsp90-TH modulator reduces the interaction between Hsp90 and TH.
